(19) United States
(12) Patent Application Publication
(10) Pub. No.: US 2020/0027557 A1
(43) Pub. Date: Jan. 23, 2020

(54) MULTIMODAL MODELING SYSTEMS AND METHODS FOR PREDICTING AND MANAGING DEMENTIA RISK FOR INDIVIDUALS

(71) Applicant: Human Longevity, Inc., San Diego, CA (US)

(72) Inventors: David Stanley Karow, San Diego, CA (US); Naisha Shah, San Diego, CA (US); Christine Menking Swisher, San Diego, CA (US); Natalie Marie Schenker-Ahmed, San Diego, CA (US); Peter Garst, San Diego, CA (US); Ilan Shomorony, San Diego, CA (US)

(21) Appl. No.: 16/288,049

(22) Filed: Feb. 27, 2019

Related U.S. Application Data
(60) Provisional application No. 62/731,070, filed on Sep. 13, 2018.
(60) Provisional application No. 62/636,794, filed on Feb. 28, 2018.

(51) Int. Cl. G16H 50/20; G16H 50/30; G16H 50/50; G16B 20/20; G16B 40/00

(52) U.S. Cl. CPC G16H 50/20; G16H 50/30; G16B 40/00; G16B 20/20; G16H 50/50

(57) ABSTRACT

The disclosure relates to systems, software and methods for diagnosis or prognosis of subjects for dementia, including, classification and treatment of subjects who have been diagnosed with or deemed at risk of dementia. The methods are based, in part, on the multimodal analysis of a plurality of features, e.g., genetic features such as SNPs or chromosome regions, including, loci or genes related thereto and structural brain features such as MRI images of brain or brain regions.

### CROSS-REFERENCE TO RELATED APPLICATIONS

The present application claims the benefit of priority under 35 U.S.C. § 119 from U.S. Provisional Patent Application No. 62/731,070, filed Sep. 13, 2018, and U.S. Provisional Patent Application No. 62/636,794, filed Feb. 28, 2018, The disclosures of which are hereby incorporated by reference in their entirety as set forth in full.

### FIELD

The embodiments disclosed herein are generally directed towards systems and methods for predicting and managing dementia risk for individuals. More specifically, there is a need for systems and methods for applying multimodal modeling techniques to make precise dementia risk predictions for individuals and identifying actionable risk factors for the same.

### BACKGROUND

Dementia is a prevalent condition, affecting 5-7% of people aged 60 years and older, and a leading cause of disability in people aged 60 years and older globally. Dementia is a clinical syndrome caused by brain damage and characterized by progressive deterioration in cognitive ability and capacity for independent living and functioning. It is considered a major global health problem. Since no cure for dementia currently exists, there is increasing focus on risk reduction, timely diagnosis, and early intervention.

Risk factors for dementia are both modifiable as well as non-modifiable. Non-modifiable risk factors include, e.g., age, family history and genetics, gender, and incidences of one or more of the following diseases: familial Alzheimer's disease, sporadic Alzheimer's disease, Parkinson's disease, multiple sclerosis, chronic kidney disease, HIV, Down syndrome and other learning disabilities. Modifiable risk factors include, e.g., alcohol use, obesity, diabetes, high blood pressure, high cholesterol, depression, head injuries, and lack of physical activity. However, the relationships between these actionable risk factors and cognitive health in general and dementia in particular are complex.

Current models for predicting the onset of dementia rely primarily on a single modality of data (e.g. magnetic resonance imaging, genotyping, laboratory screening for biomarkers, blood tests, demographics, cognitive testing, etc.). However, these existing models are simple but not so powerful at diagnosing or prognosticating a complex disorder such as dementia, wherein many factors may be at play. Recent research has indicated that there may be some advantages to using multiple modalities (in varying combinations) of imaging, genetic, clinical biomarkers, blood tests, demographics, cognitive testing, etc. as the combinations may provide more accurate predictive assess an individual's lifetime and short-term risk for dementia. Furthermore, it may allow for genotyping beyond a single gene, analyzing imaging features beyond a single structure, and provide a basis for further investigations (i.e., in-silico modeling) into potential ways to mitigate an individual's dementia risk factors (e.g. stress reduction, B12 supplementation, weight loss, alteration of medication regimen, etc.).

The ability to make predictions at the individual level may enable healthcare providers to provide a more personalized approach to treating dementia by modeling risk factors that can yield a personalized picture for each individual to provide actionable items that can be modified to reduce an individual's risk of progression. As such, there is a need for multimodal techniques that can provide accurate predictions of an individual's risk for dementia and provide risk management options to individuals.

### SUMMARY

In one aspect, provided herein are systems and methods for diagnosing dementia, which include multiple modalities including imaging, genetic and clinical biomarkers. The systems and methods of the disclosure address many limitations of the existing diagnostic assays and systems products by offering a comprehensive quantitative assessment for clinicians and other health professionals. The integrated risk profiling systems and methods of the present disclosure assesses risk of developing dementia by implementing a rigorous multimodal approach, which examines a subject's genetic and also phenotypic features, optionally together with other variables such as epidemiological factors. The device integrates multimodal data, is quantitative rather than qualitative, is objective rather than subjective, and also provides an option for outputting actionability (e.g., steps that can be taken to counter the increased risk). The systems and methods can be implemented in a minimally invasive manner, wherein the only invasive component is a routine blood draw. Actionability permits identification of factors that an individual may modify to improve their prognosis. Moreover, early screening may reduce or even eliminate psychological tension and even with a positive diagnosis, an at-risk patient can take steps to mitigate the risk.

In some embodiments, the disclosure relates to a computer readable medium comprising computer-executable instructions, which, when executed by a processor, cause the processor to carry out a method or a set of steps for diagnosing dementia in a subject, the method or steps comprising, a) extracting, into a diagnostic model, a plurality of features comprising (1) structural features of a brain tissue of the subject or a region thereof; (2) genetic features from the subject's biological sample; (3) optionally actionable risk features; and (4) further optionally epidemiological features; b) mathematically integrating the structural features and the genetic features in the diagnostic model to output a first integrated score; c) optionally integrating actionable risk features in the diagnostic model to output a second integrated score and/or further integrating epidemiological features in the diagnostic model to output a third integrated score and outputting a risk score based on the first, second or third integrated scores; and d) diagnosing dementia based on the risk score.

In some embodiments, the disclosure relates to a computer readable media of the foregoing or following, wherein the processor to carries out a method or a set of steps for diagnosing dementia in a subject, the method or steps comprising, a) extracting, into the diagnostic model, a plurality of features comprising the structural features, the genetic features and the actionable risk features; b) mathematically integrating the structural features and the genetic features to output a first integrated score; c) further integrating actionable risk features in the diagnostic model to output a second integrated score and outputting a risk score based on the second integrated score; and d) diagnosing dementia based on the risk score.

In some embodiments, the disclosure relates to a computer readable media of the foregoing or following, wherein the processor to carries out a method or a set of steps for diagnosing dementia in a subject, the method or steps comprising, a) extracting, into the diagnostic model, a plurality of features comprising the structural features, the genetic features, the actionable risk features, and the epidemiological features; b) mathematically integrating the structural features and the genetic features to output a first integrated score; c) further integrating actionable risk features in the diagnostic model to output a second integrated score and integrating the epidemiological features in the diagnostic model to output a third integrated score and outputting a risk score based on the third integrated score; and d) diagnosing dementia based on the risk score.

In some embodiments, the disclosure relates to a computer readable media of the foregoing or following, wherein the genetic features comprises at least 1, 2, 3, 4, 5, 6, 7, 8, 9, 10, 11, 12, 13, 14, 15, 16, 17, 18, 19, 20, 21, or all of the genetic features of Table 1, wherein the genetic features are listed in decreasing order of relevance to the risk score. In various embodiments, the relevance is the relative weight assigned to the genetic feature when calculating the risk score.

In some embodiments, the disclosure relates to a computer readable media of the foregoing or following, wherein the genetic features comprises at least 1, 2, 3, 4, 5, 6, 7, 8, 9, 10, 11, 12, 13, 14, 15, 16, 17, 18, 19, 20, or more SNPs or a locus related thereto.

In some embodiments, the disclosure relates to a computer readable media of the foregoing or following, wherein the genetic features comprises at least 1, 2, 3, 4, 5, 6, 7, 8, 9, 10, 11, 12, 13, 14, 15, 16, 17, 18, 19, 20, or all of the SNPs having the Ref SNP ID Nos. rs429358; rs11218343; rs6733839; rs6656401; rs9331896; rs4147929; rs10792832; rs17125944; rs7274581; rs983392; rs11771145; rs9271192; rs10948363; rs28834970; rs10498633; rs1476679; rs10838725; rs35349669; rs190982; rs2718058 or a locus related thereto.

In some embodiments, the disclosure relates to a computer readable media of the foregoing or following, wherein the genetic features comprises at least 1, 2, 3, 4, 5, 6, 7, 8, 9, 10, 11, 12, 13, 14, 15, 16, 17, 18, 19, 20, or all of the SNPs of Table 2 or a locus related thereto, wherein the genetic features are listed in the decreasing order of effect size.

In some embodiments, the disclosure relates to a computer readable media of the foregoing or following, wherein the genetic features comprises at least 1, 2, 3, 4, 5, 6, 7 or all of the rare SNPs having the Ref SNP ID Nos. rs202198008; rs538591288; rs148046938; rs113809142; rs201060968; rs775332895; and/or rs76763715 or a locus related thereto.

In some embodiments, the disclosure relates to a computer readable media of the foregoing or following, wherein the genetic features comprises at least 1, 2, 3, 4, 5, 6, 7 or all of the rare SNPs are selected from the SNPs of Table 3 or a locus related thereto.

In some embodiments, the disclosure relates to a computer readable media of the foregoing or following, wherein the genetic features comprise genetic variations comprising SNPs and/or CNVs the method includes calculation of a polygenic risk score.

In some embodiments, the disclosure relates to a computer readable media of the foregoing or following, wherein the polygenic risk score is calculated by summation of the number of risk alleles carried by an individual for each variant, weighted by the effect size (log2(OR)) from a genome-wide association study.

In some embodiments, the disclosure relates to a computer readable media of the foregoing or following, wherein the structural features of brain tissue comprises magnetic resonance imaging (MRI) data.

In some embodiments, the disclosure relates to a computer readable media of the foregoing or following, wherein the structural features include volume, cortical thickness, and cortical surface area, which are extracted for regions known to have an effect size greater than 1.

In some embodiments, the disclosure relates to a computer readable media of the foregoing or following, wherein the structural feature of brain tissue comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or all of the image features of Table 4.

In some embodiments, the disclosure relates to a computer readable media of the foregoing or following, wherein the structural features are integrated with genetic features using machine learning which comprises (1) a regularized linear model, (2) an ensemble model using boosted trees, or (3) a neural network (long short-term memory or LSTM).

In some embodiments, the disclosure relates to a computer readable media of the foregoing or following, wherein the mathematical integration comprises concatenation of the structural features with the genetic features using long short-term memory neural network.

In some embodiments, the disclosure relates to a computer readable media of the foregoing or following, wherein the actionable risk features comprise alcohol use, obesity, diabetes, high blood pressure, high cholesterol, vitamin B12, depression, head injuries, and lack of physical activity; preferably, high BMI, alcohol abuse, high cortisol, low vitamin B12, high medium-chain triglycerides (MCTs), elevated bilirubin, high triglyceride level, high serum uric acid, high diastolic blood pressure, and high systolic blood pressure.

In some embodiments, the disclosure relates to a computer readable media of the foregoing or following, wherein the epidemiological risk features comprise age-specific and gender-specific population incidence rates of dementia.

In some embodiments, the disclosure relates to a system for diagnosing dementia, comprising, a) a receiver for receiving a plurality of features comprising (1) structural features of a brain tissue of the subject or a region thereof; (2) genetic features from the subject's biological sample; (3) optionally actionable risk features; and (4) further optionally epidemiological features; b) a first integrator for integrating structural features and genetic features to output a first integrated score; c) an optional second integrator for integrating actionable risk features in the diagnostic model to output a second integrated score and a further optional third integrator for integrating the epidemiological features in the diagnostic model to output a third integrated score; and d) a scorer for determining a risk of dementia based on the first, second or third integrated score, wherein the risk score is used to diagnose dementia.

In some embodiments, the disclosure relates to a system of the foregoing or the following, which comprises the second integrator.

In some embodiments, the disclosure relates to a system of the foregoing or the following, which comprises the second integrator and the third integrator.

In some embodiments, the disclosure relates to a system of the foregoing or the following, which further comprises (e) a reporter which generates a summary report of the subject's overall risk for developing dementia in the subject's lifetime and lists all the contributing factors to the risk.

In some embodiments, the disclosure relates to a method for diagnosing dementia in a subject, comprising, a) extracting, into a diagnostic model, a plurality of features comprising (1) structural features of a brain tissue of the subject or a region thereof; (2) genetic features from the subject's biological sample; (3) optionally actionable risk features; and (4) further optionally epidemiological features; b) mathematically integrating the structural features and the genetic features in the diagnostic model to output a first integrated score; c) optionally integrating actionable risk features in the diagnostic model to output a second integrated score and/or further integrating epidemiological features in the diagnostic model to output a third integrated score and outputting a risk score based on the first, second or third integrated scores; and d) diagnosing dementia based on the risk score.

In some embodiments, the disclosure relates to a method for diagnosing dementia according to the foregoing or following, comprising, a) extracting, into the diagnostic model, a plurality of features comprising the structural features, the genetic features and the actionable risk features; b) mathematically integrating the structural features and the genetic features to output a first integrated score; c) further integrating actionable risk features in the diagnostic model to output a second integrated score and outputting a risk score based on the second integrated score; and d) diagnosing dementia based on the risk score.

In some embodiments, the disclosure relates to a method for diagnosing dementia according to the foregoing or following, comprising, a) extracting, into the diagnostic model, a plurality of features comprising the structural features, the genetic features, the actionable risk features, and the epidemiological features; b) mathematically integrating the structural features and the genetic features to output a first integrated score; c) further integrating actionable risk features in the diagnostic model to output a second integrated score and integrating the epidemiological features in the diagnostic model to output a third integrated score and outputting a risk score based on the third integrated score; and d) diagnosing dementia based on the risk score.

In some embodiments, the disclosure relates to a method for diagnosing dementia according to the foregoing or following, wherein the genetic features comprises at least 1, 2, 3, 4, 5, 6, 7, 8, 9, 10, 11, 12, 13, 14, 15, 16, 17, 18, 19, 20, 21, or all of the genetic features of Table 1, wherein the genetic features are listed in decreasing order of relevance to the risk score. In various embodiments, the relevance is the relative weight assigned to the genetic feature when calculating the risk score.

In some embodiments, the disclosure relates to a method for diagnosing dementia according to the foregoing or following, wherein the genetic features comprises at least 1, 2, 3, 4, 5, 6, 7, 8, 9, 10, 11, 12, 13, 14, 15, 16, 17, 18, 19, 20, or more SNPs or a locus related thereto.

In some embodiments, the disclosure relates to a method for diagnosing dementia according to the foregoing or following, wherein the genetic features comprises at least 1, 2, 3, 4, 5, 6, 7, 8, 9, 10, 11, 12, 13, 14, 15, 16, 17, 18, 19, 20, or all of the SNPs having the Ref SNP ID Nos. rs429358; rs11218343; rs6733839; rs6656401; rs9331896; rs4147929; rs10792832; rs17125944; rs7274581; rs983392; rs11771145; rs9271192; rs10948363; rs28834970; rs10498633; rs1476679; rs10838725; rs35349669; rs190982; rs2718058 or a locus related thereto.

In some embodiments, the disclosure relates to a method for diagnosing dementia according to the foregoing or following, wherein the genetic features comprises at least 1, 2, 3, 4, 5, 6, 7, 8, 9, 10, 11, 12, 13, 14, 15, 16, 17, 18, 19, 20, or all of the SNPs of Table 2 or a locus related thereto, wherein the genetic features are listed in decreasing order of relevance to the risk score. In various embodiments, the relevance is the relative weight assigned to the genetic feature when calculating the risk score.

In some embodiments, the disclosure relates to a method for diagnosing dementia according to the foregoing or following, wherein the genetic features comprises at least 1, 2, 3, 4, 5, 6, 7 or all of the rare SNPs having the Ref SNP ID Nos. rs202198008; rs538591288; rs148046938; rs113809142; rs201060968; rs775332895; and/or rs76763715 or a locus related thereto.

In some embodiments, the disclosure relates to a method for diagnosing dementia according to the foregoing or following, wherein the genetic features comprises at least 1, 2, 3, 4, 5, 6, 7 or all of the rare SNPs are selected from the SNPs of Table 3 or a locus related thereto.

In some embodiments, the disclosure relates to a method for diagnosing dementia according to the foregoing or following, wherein the genetic features comprise genetic variations comprising SNPs and/or CNVs the method includes calculation of a polygenic risk score.

In some embodiments, the disclosure relates to a method for diagnosing dementia according to the foregoing or following, wherein the polygenic risk score is calculated by summation of the number of risk alleles carried by an individual for each variant, weighted by the effect size (log2(OR)) from a genome-wide association study.

In some embodiments, the disclosure relates to a method for diagnosing dementia according to the foregoing or following, wherein the structural features of brain tissue comprises magnetic resonance imaging (MRI) data.

In some embodiments, the disclosure relates to a method for diagnosing dementia according to the foregoing or following, wherein the structural features include volume, cortical thickness, and cortical surface area, which are extracted for regions known to have an effect size greater than 1.

In some embodiments, the disclosure relates to a method for diagnosing dementia according to the foregoing or following, wherein the structural feature of brain tissue comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or all of the image features of Table 4.

In some embodiments, the disclosure relates to a method for diagnosing dementia according to the foregoing or following, wherein the structural features are integrated with genetic features using machine learning which comprises (1) a regularized linear model, (2) an ensemble model using boosted trees, or (3) a neural network (long short-term memory or LSTM).

In some embodiments, the disclosure relates to a method for diagnosing dementia according to the foregoing or following, wherein the mathematical integration comprises concatenation of the structural features with the genetic features using long short-term memory neural network.

In some embodiments, the disclosure relates to a method for diagnosing dementia according to the foregoing or following, wherein the actionable risk features comprise alcohol use, obesity, diabetes, high blood pressure, high cholesterol, vitamin B12, depression, head injuries, and lack of physical activity; preferably, high BMI, alcohol abuse, high cortisol, low vitamin B12, high medium-chain triglycerides (MCTs), elevated bilirubin, high triglyceride level, high serum uric acid, high diastolic blood pressure, and high systolic blood pressure.

In some embodiments, the disclosure relates to a method for diagnosing dementia according to the foregoing or following, wherein the epidemiological risk features comprise age-specific and gender-specific population incidence rates of dementia.

In some embodiments, the disclosure relates to a method for diagnosing dementia according to the foregoing or following, further comprising determining short-term or long-term risk; personalizing risk using annualized incidence rates; determining disease trajectory; identifying short-term risk of memory decline; and/or recommending an action with a recommender.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims.

FIG. 1 shows coronal, sagittal, and axial cross-sections through a patient's brain with volumetric segmentation overlaid on the structural T1-weighted MR images.

FIG. 2 shows surface area reconstruction of lateral cortical surface of a patient's brain with labeled and colorized regions. Areas with morphometrics reported are labeled and shown in yellow.

FIG. 3 shows surface area reconstruction of medial cortical surface of a patient's brain with labeled and colorized regions. Areas with morphometrics reported are labeled and shown in yellow.

FIG. 4A-4B show multimodality models for the prediction of dementia. FIG. 4A shows schematic of feature extraction from structural MRI, genetics, and modifiable risk factors derived from electronic medical records. These features are utilized in three types of models to assess an individual's risk. FIG. 4B shows outputs for the following three model types to provide a more complete picture of an individual's risk: personalized life-time risk combining population-based incidence rates and genotype-phenotype to determine the instantaneous risk for developing dementia, based on gender and age; cumulative short-term risk with in silico modification of actionable risk factors; disease progression trajectory via long short-term memory network for the prediction of the rate, onset and severity of decline with in silico modification of actionable risk factors (BP, medication, dosage).

FIG. 5A-5F shows that a combination of MRI and genetic evaluation improves the performance of disease prediction models over genetics alone. Shown are comparative analysis of the performance of the combined model to a polygenic score from genome-wide association study (GWAS), scores based on MRI imaging features, as well as the most widely used genetic (APOE4) and imaging (hippocampal occupancy) biomarkers. FIG. 5A shows Receiver Operator Curves (ROC) for personalized lifetime risk with a regularized generalized linear model with Elastic net for feature selection. FIG. 5B shows ROC for cumulative short-term risk within three years for all validation data. FIG. 5C shows ROC for only negative examples and those that transition after baseline. FIG. 5D shows model performance, as measured by area under the curve (AUC) with time, for cumulative short-term risk. FIG. 5E shows AUC ROC comparisons for within year and with three years for all validation data. FIG. 5F shows AUC ROC comparisons for within year and with three years for only negative examples and those that transition after baseline.

FIG. 6A-6D show in silico modification of actionable risk factors alters disease risk. FIG. 6A shows subtypes from multivariate survival model of disease progression shows that individuals with low, high, and normal BMI have statistically significant estimate of progression free survival. FIG. 6B shows feature importance and coverage for short-term risk model. FIG. 6C shows example of BMI inclusion in risk for in the ensemble of decision trees. Model learns AHA that BMI>25 increases risk for subset of individuals. FIG. 6D shows improvement of the model with the addition of actionable risk factors for both the short-term and long-term prognostication. The blue bars show MRI features of Table 4, in decreasing importance.

FIG. 7A-7B show cross-validation cumulative short-term risk prediction, based on ROC curves, at year three. FIG. 7A shows ROC curve of all validation data at year three. FIG. 7B shows ROC curve of validation data without dementia at baseline at year three.

FIG. 8A and 8B show risk assessment using a model that combines image features along with genetic features (MRI+GWAS) versus image features alone (MRI). FIG. 8A shows relative hazards computed by the CPH model t months prior to the “event” (either onset of Dementia or leaving the study without ever transitioning). FIG. 8B shows AUC for the task of classifying individuals that will have onset of Dementia, when considering only individuals that will either transition to Dementia in t months or leave the study in t months or more without transitioning.

FIG. 9A-9B shows features of models used to classify cognitive decline within N time frame. FIG. 9A shows model parameters. FIG. 9B shows the classification criteria for cognitive decline is defined with positive label as a change in disease state from normal to MCI or MCI to dementia.

FIG. 10A-10B show results of cross-validation of short-term memory decline. FIG. 10A shows a fivefold cross-validation ROC curves of short-term risk of cognitive decline within one year, two, three, and four years using MRI features, genetic risk scores, and demographics using ensemble of gradient boosted decision trees. FIG. 10B shows comparisons of five-fold cross-validation in other model types.

FIG. 11A-11C show results of studies of decline in memory. FIG. 11A shows ROC AUC comparison with widely used biomarkers (APOE4 status and Hippocampal Occupancy) in the short-term risk of cognitive decline within one year, two years, three and four years. FIG. 11B shows comparison of model performance by mean ROC AUCs with five-fold cross validation in models with and without MRI features and cognitive tests. FIG. 11C shows mean ROC AUCs with five-fold cross validation of cognitive decline within one year, two years, three and four years. For FIG. 11B and FIG. 11C all hyperparameters were held constant for all years (e.g. learning rate, number of iterations, depth, gamma, lambda, etc) to ensure a fair comparison, which results in a slightly reduced performance than the optimized MRI+genetics models and the MRI+genetics+cognitive models for each year.

FIG. 12A-12C show schematic for recommender: FIG. 12A shows risk factors are modified and then fed through the model. Actionable recommendations are constrained to outputs that are supported by medical literature and that are feasible and safe within a 1-year time frame. Output can be either personalized action plan via the set of changes that result in the maximum reduction in risk (shown in FIG. 12B) or personalized interactive projector (shown in FIG. 12C).

FIG. 13 shows a workflow of the disclosure. ML=machine learning.

FIG. 14 shows a representative system of the disclosure.

FIG. 15A-15E show representative reports generated by the methods and systems of the disclosure. FIG. 15A shows a report of a subject at high risk (e.g., 10× x risk compared to normal) based on genetic features alone (e.g., APOE allele e4/e4, optionally with rare SNPs in RAB10 and/or APP). A chart of annualized incidence rate with age is presented. A table showing risk of dementia with age is presented, along with a summary of genetic profile of the subject. FIG. 15B shows a report of the subject based on quantitative imaging (hippocampal volume and/or hippocampal occupancy score). A table of results and a summary of results is provided, placing the subject at low risk. FIG. 15C shows a report of the subject based on quantitative imaging (average cortex thickness and/or entorhinal cortex thickness of the left and right medial surfaces). A table of results containing information about surface area and/or thickness of various medial regions is provided, placing the subject at low risk. FIG. 15D shows a report of the subject based on quantitative imaging (average cortex thickness and/or entorhinal cortex thickness of the left and right lateral surfaces). A table of results containing information about surface area and/or thickness of various lateral regions is provided, placing the subject at low risk. FIG. 15D shows that integrating the structural features, as obtained via MRI imaging (FIG. 15B-15D) with the genetic features, as obtained using allele and/or SNP analysis (FIG. 15A), places the subject at mild risk (e.g., 4× risk compared to normal). A recommender provides an action plan to reduce this risk to normal levels, e.g., by reducing BMI to less than 25. FIG. 15E shows a summary of the combined genetic and MRI reports.

FIG. 16 shows a schematic diagram of the computer system of the disclosure.

### DETAILED DESCRIPTION

The present disclosure provides various exemplary embodiments of systems and methods for applying multimodal modeling techniques to make precise dementia risk predictions for individuals and identifying actionable risk factors for the same. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

Certain Definitions

Unless otherwise defined, all technical terms used herein have the same meaning as commonly understood by one of ordinary skill in the art to which this invention belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

As used herein, the term “individual” refers to a human individual, unless otherwise specified.

As used herein, the term “dementia” as used herein relates to a condition which can be characterized as a loss, usually progressive, of cognitive and intellectual functions, without impairment of perception or consciousness caused by a variety of disorders including severe infections and toxins, but most commonly associated with structural brain disease. Characterized by disorientation, impaired memory, judgment and intellect and a shallow labile affect. The term “dementia” includes, but is not restricted to AIDS dementia, Alzheimer dementia, presenile dementia, senile dementia, catatonic dementia, dialysis dementia (dialysis encephalopathy syndrome), epileptic dementia, hebephrenic dementia, Lewy body dementia (diffuse Lewy body disease), multi-infarct dementia (vascular dementia), paralytic dementia, posttraumatic dementia, dementia praecox, primary dementia, toxic dementia and vascular dementia. “Dementia” may include mild-cognitive impairment.

As used herein, “a symptom associated with dementia” includes, but is not limited to, memory complaint by subject or a partner; abnormal memory function (education adjusted cutoff on the logical memory II subscale); mini-mental state exam score between 24-40 (preferably between 20-26); clinical dementia rating of about 0.5 (or more); memory box score of at least 0.5; Alzheimer's Association's NINCDS/ADRDA criteria for probable AD; or a combination thereof.

As used herein, the term “diagnosis” refers to methods by which a determination can be made as to whether a subject is likely to be suffering from a given disease or condition, including but not limited symptoms associated with the disease or condition. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms, e.g., memory loss; phenotype; genotype; or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given characteristic.

The term “extract” used in the present invention means to obtain data to determine a marker (e.g., a genetic marker such as SNP or an image marker such as a pixel) at a specific time in a predetermined period. With respect to image data, the term may include two-dimensional or three-dimensional representations.

The term “two-dimensional” or “three-dimensional” in the context of image data means expression of the image in terms of the coordinate positions by using two coordinates or three coordinates. A “two-dimensional image” in the present invention includes a cross section image which is acquired by imaging a certain cross section, as well as a two-dimensional projected image which is acquired by projecting three-dimensional image data obtained by imaging a subject.

The term “brain tissue” as used herein refers to the brain or any portion of the brain, including, but not limited to, whole brain, parenchyma, ventricles, intracranial spaces, intraventricular space, and intravascular space. The term includes neural pathways, neuro-endocrine systems, neuro-vascular systems and dural-meningial systems.

As used herein, the term “brain region” includes, but is not limited to, hindbrain (rhombencephalon)(includes myelencephalon or metencephalon); midbrain (mesencephalon); forebrain (prosencephalon) comprising diencephalon (includes epithalamus; third ventricle; thalamus; hypothalamus (limbic system); subthalamus; and pituitary gland) and telencephalon (cerebrum) comprising white matter, subcortical regions, rhinencephalon (paleopallium), and cerebral cortex (neopallium). The term additionally includes sub-regions of the aforementioned anatomical regions.

As used herein, the term “marker” refers to a characteristic that can be objectively measured as an indicator of normal biological processes, pathogenic processes (e.g., Alzheimer's) or a response to an intervention, e.g., treatment with an anti-dementia agent (e.g., cholinesterase inhibitors (donepezil, rivastigmate, galantamine) and memantine). Representative types of markers include, for example, genomic markers, structural markers, actionable markers, epedimiological markers, or a combination thereof. Genomic markers include, e.g., molecular changes in the structure (e.g., sequence) or number of the genetic feature, comprising, e.g., polymorphisms, gene mutations, gene duplications, or a plurality of differences, such as somatic alterations in DNA, copy number variations, tandem repeats, or a combination thereof. Structural markers include image data of the tissue or region of interest, e.g., whole brain or an affected region thereof (AD initially affects brain regions involved in memory, including the entorhinal cortex and hippocampus and later affects areas in the cerebral cortex responsible for language, reasoning, and social behavior).

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by intemucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The term “genetic feature” refers to a property of a genome or an expression product thereof (e.g., an mRNA transcriptome or a polypeptide proteome). The term encompasses positions in a genome (e.g., chromosome) as well as changes therein (e.g., a variant genome). Preferably, the genetic feature includes variant nucleic acids, e.g., mutations, SNPs, CNVs, STRs, or a combination thereof compared to a reference sample. Particularly, the variations are in the coding region of the nucleic acids, especially in the exomes. The variant nucleic acids preferably encode for an altered protein product, e.g., a protein product whose amino acid composition or length or both is different from a reference (e.g., wild-type) polypeptide product. “Genetic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

As used herein, the term “single nucleotide polymorphism” or “single nucleotide variation” (“SNP” or “SNV”) in reference to a mutation refers to a difference of at least one nucleotide in a sequence in comparison to another sequence. The term “copy number variation” or “CNV” refers to a comparative numerical change in the presence or absence/gain or loss, of gene fragments having the same nucleotide sequence.

The term “indel” as used herein, and generally in the art, refers to a location on a genome where one or more bases are present in one allele, with no bases present in another allele. Insertions or deletions are distinct from an evolutionary point of view, but during analysis such as described herein, they are often not distinguished as an insertion in one allele is equivalent to a deletion in the other allele. Thus the term indel is to refer to the location of the insertion/deletion between two alleles.

“Structural variants” involve changes in some parts of the chromosomes instead of changes in the number of chromosomes or sets of chromosomes in the genome. There are four common types of mutations which result in structural variants: deletions and insertions, for example duplications (involving a change in the amount of DNA in a chromosome, loss and gain of genetic material, respectively), inversions (involving a change in the arrangement of a chromosomal segment) and translocations (involving a change in the location of a chromosomal segment which can give rise to gene fusions). In the present invention, the term “structural variant” includes loss of genetic material, a gain of genetic material, a translocation, a gene fusion and combinations thereof.

As used herein, the term “variation” refers to a change or deviation. In reference to nucleic acid, a variation refers to a difference(s) or a change(s) between DNA nucleotide sequences, including differences in copy number (CNVs). This actual difference in nucleotides between DNA sequences may be an SNP, and/or a change in a DNA sequence, e.g., fusion, deletion, addition, repeats, etc., observed when a sequence is compared to a reference, such as, e.g., germline DNA (gDNA) or a reference human genome HG38 sequence. Information on short genetic variations can be obtained using NCBI's SNP database (dbSNP) using Ref SNP (rs) numbers. Information on large structural variations, e.g., insertions, deletions, duplications, inversions, mobile elements, and translocations can be obtained using NCBI's variation database (dbVar) using an NCBI (nsv) or EBI (esv) reference number.

A variation can be “rare” “low frequency” or “common.” Generally, common variants have a minor allele frequency (MAF) that is greater than 5% and usually exert a very weak effect or association with the phenotype (e.g., a disease) of interest. Low-frequency variants typically have a MAF of about 1%-5%. In contrast, rare variants typically have a MAF<1%, or even <0.2% and may exert a small to modest effect or association with the phenotype (e.g., a disease) of interest.

The term “polygenic” as used herein refers to association with multiple genetic features, e.g., mutations, polymorphisms, CNVs, indels, duplications, or translocations, in more than a single gene. Polygenic traits usually include complex diseases, disorders, syndromes that are caused by dysfunction in two or more genes and may also include non-pathological characteristics associated with the interaction of two or more genes. The term is contrasted with “monogenic” which refers to association of a trait, normal or pathological, with a single genetic feature. Monogenic traits usually include diseases caused by a dysfunction in a single gene (e.g., sickle cell anemia). Monogenic traits also include non-pathological characteristics (e.g., presence or absence of cell surface molecules on a specific cell type).

As used herein, the term “missense mutation” refers to a change in the DNA sequence that changes a codon in the MRNA that is normally translated as one amino acid into a codon that is translated as a different amino acid. Some but not all missense mutations result in a non-functional gene-product. Some missense mutations may also result in a gain of function. A selection method may be used to find those missense mutations that substantially affect the protein function.

As used herein, the term “loss-of-function (LoF) mutation” or “inactivating mutation” refers to mutations which result in partial or complete inactivation of the gene product. The term includes “amorphic mutation” which refers to instances wherein an allele has a complete loss of function (null allele). In contrast, “gain-of-function (GoF) mutations” or “activating mutations” refers to mutations which enhance activity of the protein product or which result in a wholly different (and abnormal) activity of the protein.

A “locus” (plural “loci”) corresponds to an identified location in a genome, and can span a single base or a sequential series of multiple bases. A locus is typically identified by using an identifier value or a range of identifier values with respect to a reference genome and/or a chromosome thereof. A “heterozygous locus” (also referred to as a “het”) is a locus in a genome, where the two copies of a chromosome do not have the same sequence. These different sequences at a locus are called “alleles”. A het can be a single-nucleotide polymorphism (SNP) if the reference genome location has two alleles that differ by a single base. A “het” can also be a reference genome location where there is an insertion or a deletion (collectively referred to as an “indel”) of one or more nucleotides or one or more tandem repeats. A “homozygous locus” is a locus in a reference or a baseline genome, where the two copies of a chromosome have the same allele. “Haplotype” of a chromosome refers to whether the chromosome is present once or twice in a genome. A “region” in a genome may include one or more loci.

As used herein, the term “germline DNA” or “gDNA” refers to DNA isolated or extracted from a subject's germline cells, e.g., peripheral mononuclear blood cells, including lymphocytes that are in turn obtained from circulating blood.

The term “control,” as used herein, refers to a reference for a test sample, such as control DNA isolated from peripheral mononuclear blood cells and lymphocytes, where these cells are not cancer cells, and the like. A “reference sample,” as used herein, refers to a sample of tissue or cells that may or may not have cancer that are used for comparisons. Thus a “reference” sample thereby provides a basis to which another sample, for example plasma sample containing markers, e.g., exomic markers can be compared. In contrast, a “test sample” refers to a sample compared to a reference sample or control sample. In some embodiments, the reference sample or control may comprise a reference assembly.

The term “reference assembly” refers to a digital nucleic acid sequence database, such as the human genome (HG38) database containing HG38 assembly sequences. The gateway can be accessed through the Human (Homo sapiens) University of California Santa Cruz Genome Browser Gateway via the web at genome(dot)ucsc(dot)edu. Alternately, the reference assembly may refer to the Genome Reference Consortium's Human Genomic Assembly (Build #38; Assembled: June, 2017), which is accessible on the internet via the U.S. NCBI website.

As used herein, the term “sequencing” or “sequence” as a verb refers to a process whereby the nucleotide sequence of DNA, or order of nucleotides, is determined, such as a nucleotide order AGTCC, etc. The term “sequence” as a noun refers to the actual nucleotide sequence obtained from sequencing; for example, DNA having the sequence AGTCC. Wherein the “sequence” is provided and/or received in digital form, e.g., in a disk or remotely via a server, “sequencing” may refer to a collection of DNA that is propagated, manipulated and/or analyzed using the methods and/or systems of the disclosure.

The term “sequencing run” refers to any step or portion of a sequencing experiment performed to determine some information relating to at least one biomolecule (e.g., nucleic acid molecule).

The term “whole genome sequencing” or “WGS” refers to a laboratory process that determines the DNA sequence of each DNA strand in a sample. The resulting sequences may be referred to as “raw sequencing data” or “read.” As used herein, a read is a “mappable” read when the sequence has similarity to a region of a reference chromosomal DNA sequence. The term “mappable” may refer to areas that show similarity to and thus “mapped” to a reference sequence, for example, a segment of cfDNA showing similarity to reference sequence in a database, for example, cfDNA having a high percentage of similarity to human chromosomal region 8q248q24.3 in the human genome (HG38) database, is a “mappable read.”

In addition to “WGS,” the genomic compendiums may be obtained using targeted sequencing. In contrast to WGS, the term “targeted sequencing,” as used herein, refers to a laboratory process that determines the DNA sequence of chosen DNA loci or genes in a sample, for example sequencing a chosen group of cancer-related genes or markers (e.g., a target). In this context, the term “target sequence” herein refers to a selected target polynucleotide, e.g., a sequence present in a cfDNA molecule, whose presence, amount, and/or nucleotide sequence, or changes therein, are desired to be determined. Target sequences are interrogated for the presence or absence of a somatic mutation. The target polynucleotide can be a region of gene associated with a disease, e.g., cancer. In some embodiments, the region is an exon.

As used herein the term “whole exome sequencing” refers to selective sequencing of coding regions of the DNA genome. The targeted exome is usually the portion of the DNA that translate into proteins, however regions of the exome that do not translate into proteins may also be included within the sequence. The robust approach to sequencing the complete coding region (exome) can be clinically relevant in genetic diagnosis due to the current understanding of functional consequences in sequence variation, by identifying the functional variation that is responsible for both Mendelian and common diseases without the high costs associated with a high coverage whole-genome sequencing while maintaining high coverage in sequence depth. See, Ng et al., Nature 461, 272-276, 2009 and Choi et al., PNAS USA 106, 19096-19101, 2009.

As used herein the term “whole transcriptome sequencing” refers to determining the expression of all RNA molecules including messenger RNA (mRNA), ribosomal RNA (rRNA), transfer RNA (tRNA), and non-coding RNA. Whole transcriptome sequencing can be done with a variety of platforms for example, the Genome Analyzer (Illumina, Inc., San Diego, Calif., USA) and the SOLID™ Sequencing System (Life Technologies, Carlsbad, Calif., USA). However, any platform useful for whole transcriptome sequencing may be used. The term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq includes whole transcriptome sequencing as well as target specific sequencing.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Various aspects and embodiments of the systems and methods disclosed herein employ the use of NGS technologies. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in WO 2006/084132 and U.S. Pat. Nos. 8,536,099 and 8,934,098, the entirety of each of these applications being incorporated herein by reference thereto.

Genomic variants can be identified using a variety of techniques, including, but not limited to: array-based methods (e.g., DNA microarrays, etc.), real-time/digital/quantitative PCR instrument methods and whole or targeted nucleic acid sequencing systems (e.g., NGS systems, Capillary Electrophoresis systems, etc.). With nucleic acid sequencing, coverage data can be available at single base resolution.

As used herein, the phrase “genomic region” or “genome region” denotes a region within a genome that can be defined in one of three ways—as (1) by a tagging SNP region, (2) an explicitly defined genomic region, or (3) a list of genes. For example, (1) genomic regions can be defined around any SNPs listed in HapMap. That is, a region can be defined around any named SNP using linkage disequilibrium (LD) properties. Specifically, the SNP region can start at the SNP location and proceed to the furthest neighboring SNPs in the 3′ and 5′ direction in LD (r2 >0.5). It can then proceed outwards in each direction to the nearest recombination hotspot. If no genes are in that region—the region can be expanded a set number of bases (i.e., 250 kb or more) in either direction. (2) Regions can also be explicitly defined. In that case indicate the Human Genome Assembly (e.g., hg17, hg18, etc.) that your regions are defined in. Then describe the region with four fields in order: a unique word identifier, the chromosome that the region is on, the start position (base pairs), and the end position (base pairs). (3) Regions can also be defined as a gene list. In this case for each line enter a unique word identifier, followed by the term GID. Then list each gene separated by spaces using their Entrez ID.

As used herein, the phrase “linked” refers to a region of a chromosome that is shared more frequently in family members affected by a particular disease, than expected by chance, thereby indicating that the gene or genes within the linked chromosome region contain or are associated with a marker or functional polymorphism that is correlated to the presence of, or risk of, disease. Once linkage is established, association studies (linkage disequilibrium) can be used to narrow the region of interest or to identify the risk conferring gene for Alzheimer's disease.

As used herein, the phrase “associated with” when used to refer to a marker or functional polymorphism and a particular gene means that the functional polymorphism is either within the indicated gene, or in a different physically adjacent gene on that chromosome. In general, such a physically adjacent gene is on the same chromosome and within 2 or 3 centimorgans of the named gene (i.e., within about 3 million base pairs of the named gene).

As used herein, the term “actionable risk features” includes phenotypic, lifestyle, and environmental features that can be modified. Representative examples include, but are not limited to, alcohol use (action: lower intake), obesity (action: reduce caloric intake), diabetes (action: lower sugar intake; take diabetes medication), high blood pressure (action: lower salt intake; take antihypertensive medication), high cholesterol (action: lower cholesteric food intake; take drugs such as statins), vitamin B12 (action: consume B12-rich foods), depression (action: take antidepressants), head injuries (action: reduce contact sports), and lack of physical activity (action: increase exercise); preferably, high BMI, alcohol abuse, high cortisol, low vitamin B12, high medium-chain triglycerides (MCTs), elevated bilirubin, high triglyceride level, high serum uric acid, high diastolic blood pressure (BP), and high systolic BP.

As used herein, the term “epidemiological features” include population-specific parameters of a disease of interest. The term includes, prevalence, incidence, person-time at risk, duration of disease, survival, mortality, including measures of effect (e.g., risk ratio, rate ratio, odds ratio) in a population or sub-population of subjects.

As used herein, the phrase “medical imaging techniques”, “medical imaging methods” or “medical imaging systems” can denote techniques or processes for obtaining visual representations of the interior of an individual's body for clinical analysis and medical intervention, as well as visual representation of the function of some organs or tissues. Within these visual representations various imaging features can be identified and characterized to provide a structural basis for diagnosing and treating various types of diseases (e.g., dementia, cancer, cardiovascular disease, cerebrovascular disease, liver disease, etc). Examples of medical imaging techniques can include, but are not limited to, x-ray radiography, magnetic resonance imaging, ultrasound, positron emission tomography (PET), computed tomography (CT), etc.

Various aspects and embodiments of the methods and systems disclosed herein use conventional and specialized sequence alignment methods that can align a fragment sequence to a reference sequence or another fragment sequence. The fragment sequence can be obtained from a fragment library, a paired-end library, a mate-pair library, a concatenated fragment library, or another type of library that may be reflected or represented by nucleic acid sequence information including for example, RNA, DNA, and protein based sequence information. Generally, the length of the fragment sequence can be substantially less than the length of the reference sequence. The fragment sequence and the reference sequence can each include a sequence of symbols. The alignment of the fragment sequence and the reference sequence can include a limited number of mismatches between the symbols of the fragment sequence and the symbols of the reference sequence. Generally, the fragment sequence can be aligned to a portion of the reference sequence in order to minimize the number of mismatches between the fragment sequence and the reference sequence.

In various embodiments, the symbols of the fragment sequence and the reference sequence can represent the composition of biomolecules. For example, the symbols can correspond to identity of nucleotides in a nucleic acid, such as RNA or DNA, or the identity of amino acids in a protein. In some embodiments, the symbols can have a direct correlation to these subcomponents of the biomolecules. For example, each symbol can represent a single base of a polynucleotide. In other embodiments, each symbol can represent two or more adjacent subcomponent of the biomolecules, such as two adjacent bases of a polynucleotide. Additionally, the symbols can represent overlapping sets of adjacent subcomponents or distinct sets of adjacent subcomponents. For example, when each symbol represents two adjacent bases of a polynucleotide, two adjacent symbols representing overlapping sets can correspond to three bases of polynucleotide sequence, whereas two adjacent symbols representing distinct sets can represent a sequence of four bases. Further, the symbols can correspond directly to the subcomponents, such as nucleotides, or they can correspond to a color call or other indirect measure of the subcomponents. For example, the symbols can correspond to an incorporation or non-incorporation for a particular nucleotide flow.

Various embodiments of the systems and methods disclosed herein use a computer program product that can include instructions to select a contiguous portion of a fragment sequence; instructions to map the contiguous portion of the fragment sequence to a reference sequence using an approximate string matching method that produces at least one match of the contiguous portion to the reference sequence.

Various embodiments of the systems and methods disclosed herein use a system for nucleic acid sequence analysis that can include a data analysis unit. The data analysis unit can be configured to obtain a fragment sequence from a sequencing instrument, obtain a reference sequence, select a contiguous portion of the fragment sequence, and map the contiguous portion of the fragment sequence to the reference sequence using an approximate string mapping method that produces at least one match of the contiguous potion to the reference sequence.

### Multimodal Feature Analysis of an Individual's Risk for Dementia

Various aspects and embodiments are disclosed herein for applying multimodal modeling techniques to make precise dementia risk predictions for individuals and identify actionable risk factors for the same. For example, in one aspect, two or more modalities of data (e.g. medical imaging, genotyping, laboratory screening for biomarkers, blood tests, demographics, cognitive testing, etc.) are combined to predict an individual's risk for developing dementia in his/her lifetime and identify actionable risk factors (e.g., blood pressure, cortisol levels, medications, BMI, cholesterol, diet, etc.) to mitigate that risk.

In a preferred embodiment, different artificial intelligence and/or machine learning techniques are used to predict an individual's risk for developing dementia using genetic features data (obtained thru whole genome sequencing) known to be associated with Alzheimer's risk. In certain embodiments, the genetic features comprise at least 1, 2, 3, 4, 5, 6, 7, 8, 9, 10, 11, 12, 13, 14, 15, 16, 17, 18, 19, 20, 21, or all of the genetic features of Table 1:

TABLE 1
List of genetic features associated with dementia,
in the order of relevance to Alzheimer's risk

| Chromosome | Region_Start | Region_Stop |
|---|---|---|
| chr19 | 43908684 | 45908684 |
| chr2 | 25135287 | 27135287 |
| chr11 | 120564878 | 122564878 |
| chr2 | 126135234 | 128135234 |
| chr1 | 206518704 | 208518704 |
| chr8 | 26610169 | 28610169 |
| chr19 | 63444 | 2063444 |
| chr11 | 85156833 | 87156833 |
| chr14 | 51933911 | 53933911 |
| chr20 | 55443204 | 57443204 |
| chr11 | 59156035 | 61156035 |
| chr7 | 142413669 | 144413669 |
| chr6 | 31610753 | 33610753 |
| chr6 | 46520026 | 48520026 |
| chr8 | 26337604 | 28337604 |
| chr14 | 91460608 | 93460608 |
| chr7 | 99406823 | 101406823 |
| chr11 | 46536319 | 48536319 |
| chr2 | 232159830 | 234159830 |
| chr5 | 87927603 | 89927603 |
| chr7 | 36801932 | 38801932 |

Information related to the genetic features may be obtained using routine means. For instance, using University of California Santa Cruz's Genome Browser on Human (GRCh38/hg38) Assembly (assembled: DEC 2013), which s accessible on the web at genome(dot)ucsc(dot)edu/cgi-bin/hgGateway. Therein, an assembly is selected (e.g., Genome Reference Consortium Human Build 38 (GRCh38) and under the search field, the chromosome number and the region is specified (e.g., chr19:43,908,684-45,908,684).

More specifically, the genomic markers comprise at least 1, 2, 3, 4, 5, 6, 7, 8, 9, 10, 11, 12, 13, 14, 15, 16, 17, 18, 19, 20, or more SNPs or a locus related thereto. In certain embodiments, the image features comprise at least 1, 2, 3, 4, 5, 6, 7, 8, 9, 10, 11, 12, 13, 14, 15, 16, 17, 18, 19, 20, or all of the genetic markers comprising SNPs having the Ref SNP ID Nos. rs429358; rs11218343; rs6733839; rs6656401; rs9331896; rs4147929; rs10792832; rs17125944; rs7274581; rs983392; rs11771145; rs9271192; rs10948363; rs28834970; rs10498633; rs1476679; rs10838725; rs35349669; rs190982; rs2718058 or a locus related thereto. Preferably, the SNPs are selected from the SNPs of Table 2 or a locus related thereto:

TABLE 2
List of SNPs, ranked in decreasing order of effect size.

| rsID | Chromosome | Position |
|---|---|---|
| rs429358 | chr19 | 44908684 |
| rs11218343 | chr11 | 121564878 |
| rs6733839 | chr2 | 127135234 |
| rs6656401 | chr1 | 207518704 |
| rs9331896 | chr8 | 27610169 |
| rs4147929 | chr19 | 1063444 |
| rs10792832 | chr11 | 86156833 |
| rs17125944 | chr14 | 52933911 |
| rs7274581 | chr20 | 56443204 |
| rs983392 | chr11 | 60156035 |
| rs11771145 | chr7 | 143413669 |
| rs9271192 | chr6 | 32610753 |
| rs10948363 | chr6 | 47520026 |
| rs28834970 | chr8 | 27337604 |
| rs10498633 | chr14 | 92460608 |
| rs1476679 | chr7 | 100406823 |
| rs10838725 | chr11 | 47536319 |
| rs35349669 | chr2 | 233159830 |
| rs190982 | chr5 | 88927603 |
| rs2718058 | chr7 | 37801932 |

In some embodiments, the genetic features that are measured additionally include one or more rare genetic markers associated with dementia. In certain embodiments, the genetic features comprise at least 1, 2, 3, 4, 5, 6, 7 or all of the rare SNPs having the Ref SNP ID Nos. rs202198008; rs538591288; rs148046938; rs113809142; rs201060968; rs775332895; and/or rs76763715 or a locus related thereto. Preferably, the rare SNPs are selected from the SNPs of Table 3 or a locus related thereto:

TABLE 3
Rare genetic markers associated with dementia

| Chromosome | Position | dbSNP | Gene | ExAC AF |
|---|---|---|---|---|
| chr21 | 26021879 | rs202198008 | APP | 0.0006 |
| chr19 | 1055908 | rs538591288 | ABCA7 | 0.000882 |
| chr19 | 15186898 | rs148046938 | NOTCH3 | 0.000701 |
| chr19 | 1056245 | rs113809142 | ABCA7 | 0.000156 |
| chr19 | 1054256 | rs201060968 | ABCA7 | 0.000518 |
| chr22 | 23767396 | rs775332895 | CHCHD10 | 0.000287 |
| chr1 | 1.55E+08 | rs76763715 | GBA | 0.00221 |

In certain embodiments, the genetic feature comprises variations in apolipoprotein E (APOE) or allele status thereof. Three model types may be used for the prediction of Alzheimer's disease (AD) based on this genetic feature—(a) life-time risk; (b) cumulative short-term risk; and (c) disease trajectory. In certain embodiments, the model predicts AD in subjects with compromised genetic features (apolipoprotein E (APOE) allele status e4/e4) but having good imaging phenotype (hippocampal occupancy score>70%). In certain embodiments, the model predicts AD in subjects with AD in subjects with compromised genetic features (e4/e4) and also having poor imaging phenotype (hippocampal occupancy score<20%).

In some embodiments, the features additionally comprise a set of imaging features data obtained from structural T1-weighted magnetic resonance imaging (MRI) images of an individual's brain. In certain embodiments, the image features comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or all of the image features of Table 4:

TABLE 4
List of image features

| SN | Image feature |
|---|---|
| 1 | Estimated Total IntraCranial Volume |
| 2 | Left hemisphere Hippocampus Volume |
| 3 | Right hemisphere Hippocampus Volume |
| 4 | Left hemisphere Lateral Ventricle Volume |
| 5 | Right hemisphere Lateral Ventricle Volume |
| 6 | Left hemisphere Inferior Lateral Ventricle Volume |
| 7 | Right hemisphere Inferior Lateral Ventricle Volume |
| 8 | Left hemisphere Amygdala Volume |
| 9 | Right hemisphere Amygdala Volume |
| 10 | Left hemisphere entorlinal Gray Volume |
| 11 | Left hemisphere entorlinal Surface Area |
| 12 | Left hemisphere entorlinal Thickness Average |
| 13 | Right hemisphere entorlinal Gray Volume |
| 14 | Right hemisphere entorlinal Surface Area |
| 15 | Right hemisphere entorlinal Thickness Average |
| 16 | Left hemisphere parahippocampal Gray Volume |
| 17 | Right hemisphere parahippocampal Gray Volume |
| 18 | Left hemisphere inferiorparietal Gray Volume |
| 19 | Left hemisphere inferiorparietal Surface Area |
| 20 | Left hemisphere inferiorparietal Thickness Average |
| 21 | Right hemisphere inferiorparietal Gray Volume |
| 22 | Right hemisphere inferiorparietal Surface Area |
| 23 | Right hemisphere inferiorparietal Thickness Average |
| 24 | Left hemisphere rostral middle frontal Gray Volume |
| 25 | Left hemisphere rostral middle frontal Surface Area |
| 26 | Left hemisphere rostral middle frontal Thickness Average |
| 27 | Right hemisphere rostral middle frontal Gray Volume |
| 28 | Right hemisphere rostral middle frontal Surface Area |
| 29 | Right hemisphere rostral middle frontal Thickness Average |
| 30 | Left hemisphere isthmuscingulate Gray Volume |
| 31 | Left hemisphere isthmuscingulate Surface Area |
| 32 | Left hemisphere isthmuscingulate Thickness Average |
| 33 | Right hemisphere isthmuscingulate Gray Volume |
| 34 | Right hemisphere isthmuscingulate Surface Area |
| 35 | Right hemisphere isthmuscingulate Thickness Average |
| 36 | Left hemisphere supramarginal Gray Volume |
| 37 | Left hemisphere supramarginal Surface Area |
| 38 | Left hemisphere supramarginal Thickness Average |
| 39 | Right hemisphere supramarginal Gray Volume |
| 40 | Right hemisphere supramarginal Surface Area |
| 41 | Right hemisphere supramarginal Thickness Average |
| 42 | Left hemisphere caudal middle frontal Gray Volume |
| 43 | Left hemisphere caudal middle frontal Surface Area |
| 44 | Left hemisphere caudal middle frontal Thickness Average |
| 45 | Right hemisphere caudal middle frontal Gray Volume |
| 46 | Right hemisphere caudal middle frontal Surface Area |
| 47 | Right hemisphere caudal middle frontal Thickness Average |
| 48 | Left hemisphere fusiform Gray Volume |
| 49 | Left hemisphere fusiform Surface Area |
| 50 | Left hemisphere fusiform Thickness Average |
| 51 | Right hemisphere fusiform Gray Volume |
| 52 | Right hemisphere fusiform Surface Area |
| 53 | Right hemisphere fusiform Thickness Average |
| 54 | Right hemisphere middle temporal Gray Volume |
| 55 | Left hemisphere middle temporal Gray Volume |
| 56 | Right hemisphere middle temporal Surface Area |
| 57 | Left hemisphere middle temporal Surface Area |
| 58 | Right hemisphere middle temporal Thickness Average |
| 59 | Left hemisphere middle temporal Thickness Average |
| 60 | Right hemisphere inferior temporal Gray Volume |
| 61 | Left hemisphere inferior temporal Gray Volume |
| 62 | Right hemisphere inferior temporal Surface Area |
| 63 | Left hemisphere inferior temporal Surface Area |
| 64 | Right hemisphere inferior temporal Thickness Average |
| 65 | Left hemisphere inferior temporal Thickness Average |
| 66 | Right hemisphere parahippocampal Surface Area |
| 67 | Left hemisphere parahippocampal Surface Area |
| 68 | Right hemisphere parahippocampal Thickness Average |
| 69 | Left hemisphere parahippocampal Thickness Average |
| 70 | Right hemisphere precuneus Gray Volume |
| 71 | Left hemisphere precuneus Gray Volume |
| 72 | Right hemisphere precuneus Surface Area |
| 73 | Left hemisphere precuneus Surface Area |
| 74 | Right hemisphere precuneus Thickness Average |
| 75 | Left hemisphere precuneus Thickness Average |
| 76 | Left hemisphere Hippocampal occupancy |
| 77 | Right hemisphere Hippocampal occupancy |
| 78 | White Matter hypointensities from T1W imaging |
| 79 | White Matter hyperintensities from FLAIR (volume, count, location) |

These genomic and imaging features are used to train the multimodal models that predict the likelihood of an individual's progression to dementia. Examples of multimodal models vary in complexity and the approach they take to the sequential nature of the data: (1) a regularized linear model, (2) an ensemble model using boosted trees, and (3) a neural network (long short-term memory or LSTM). For all three models, respectively, using MRI and whole genome sequencing data combined improves performance (1: AUC=0.95, 2: AUC=0.92 within 4 years, 3: AUC=0.92) in the prediction of dementia progression over either MRI (1: AUC=TBD, 2: AUC=TBD, 3: AUC=TBD) or WGS (1: AUC=0.82, 2: AUC=TBD, 3: AUC=TBD) alone.

In various embodiments, in addition to features from MRI and WGS, the models also utilized features from demographics (age, gender, education) and actionable risk factors (such as blood pressure, p=TBD and BMI, p=TBD).

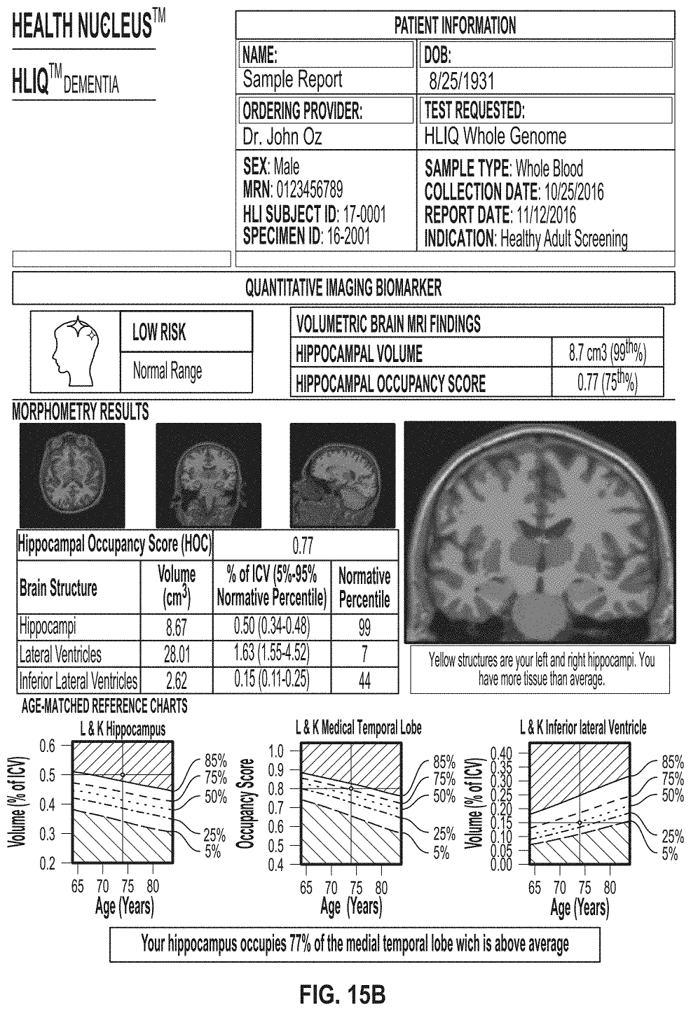

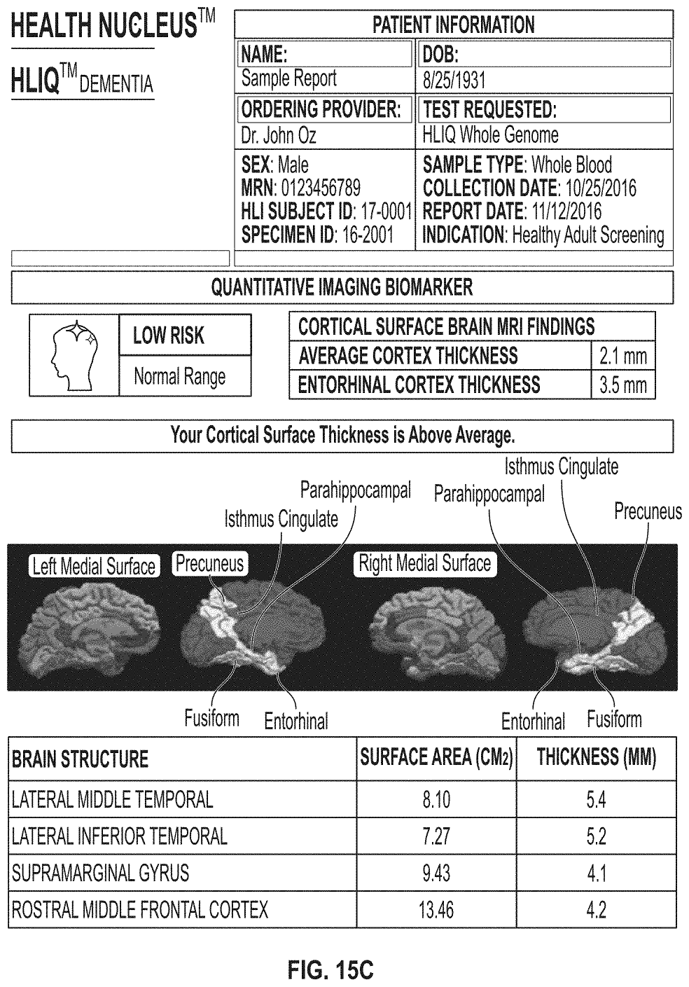

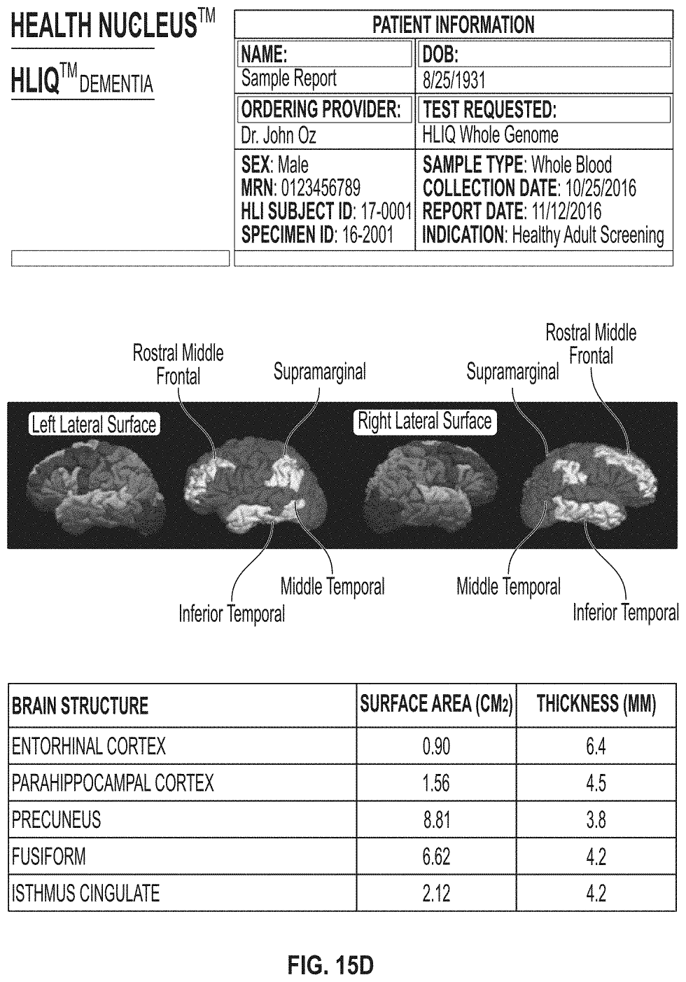

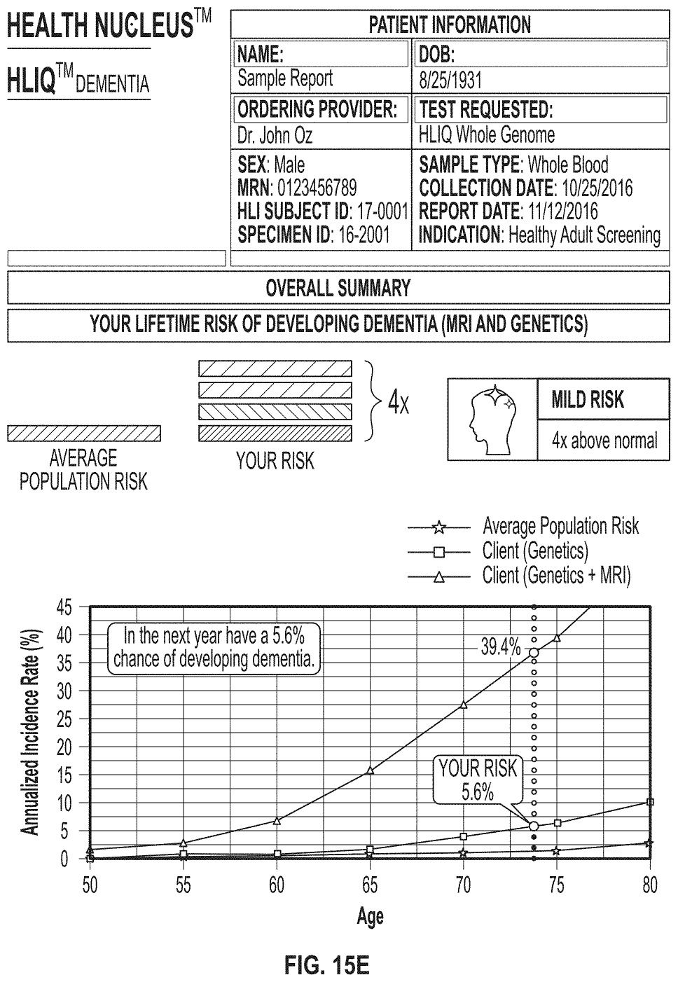

In various embodiments, after multimodal analysis of an individuals' genomic and imaging features data is complete, a report is generated that summarizes that individual's overall risk for developing dementia in his/her lifetime and all the contributing factors to that risk. Representative reports are shown in FIG. 15A (genetic report), FIG. 15B-FIG. 15D (MRI reports) and FIG. 15E (combined genetic and MRI reports).

In some embodiments, the present invention provides systems and method for computation of polygenic personalized risk scores leveraging genetic features by employing the statistical methodology described herein. For example, genetic features (e.g., single nucleotide polymorphisms (SNPs) or chromosome positions), which are associated with dementia, are leveraged to output a polygenic risk score. In some embodiments, genetic markers associated with Alzheimer's disease are identified from published genome-wide association studies (GWAS) and the polygenic score is calculated by summation of the number of risk alleles carried by an individual for each variant, weighted by the effect size (log₂(odds ratio)) from the GWAS. The higher effect size, the stronger the association between the genetic feature with the disease.

In some embodiments, the score for each individual is normalized to a reference population of matching ancestry to account of any allele frequency differences between ancestral populations.

In some embodiments, computation of polygenic risk scores leverages genetic feature and the ancestral match simultaneously. In some embodiments, computation of polygenic risk scores leverages other types of prior information. In some embodiments, genetic personalized risk scores summarize patient-level genomic variation as a single score per subject, summed over assayed gene variants.

In some embodiments, the polygenic risk score is computed as a linear or nonlinear function of the estimated statistical parameters, including mean per SNP allele effect size and/or estimates of variability. Preferably, statistical methods are utilized to obtain maximal correlation of genetic risk scores with phenotypes in de novo subject samples. In some embodiments, gene variant effect sizes below a given threshold are deleted before computing polygenic risk scores. In some embodiments, polygenic risk scores also include other biomarkers of complex phenotypes or disease diagnosis. Other biomarkers of risk include, but are not limited to, age, gender, family history of illness, etc.

Methods for Determining Short-Term Risk

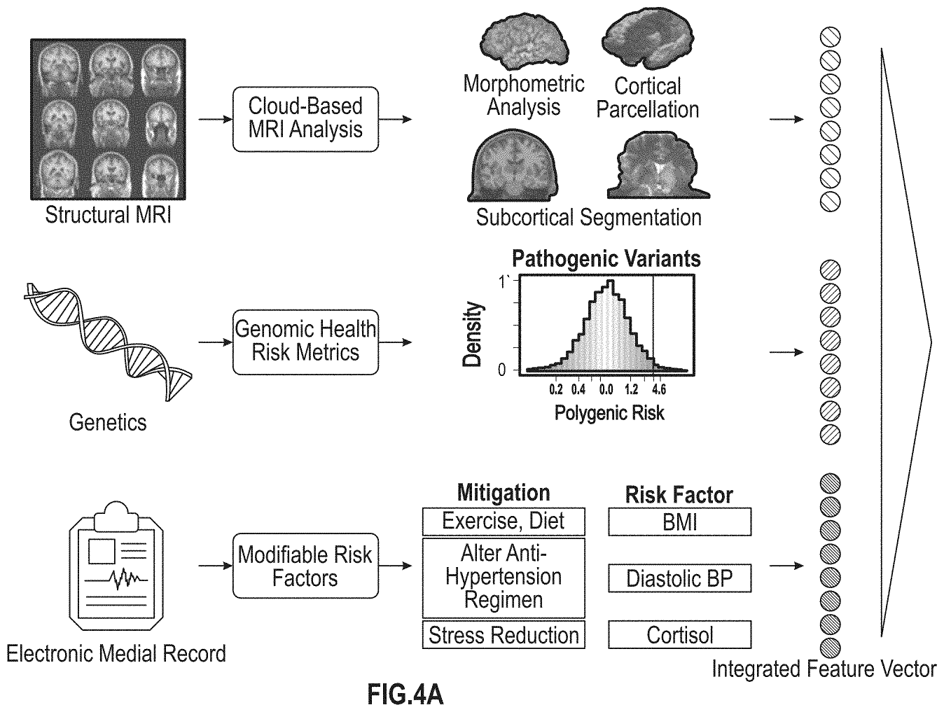

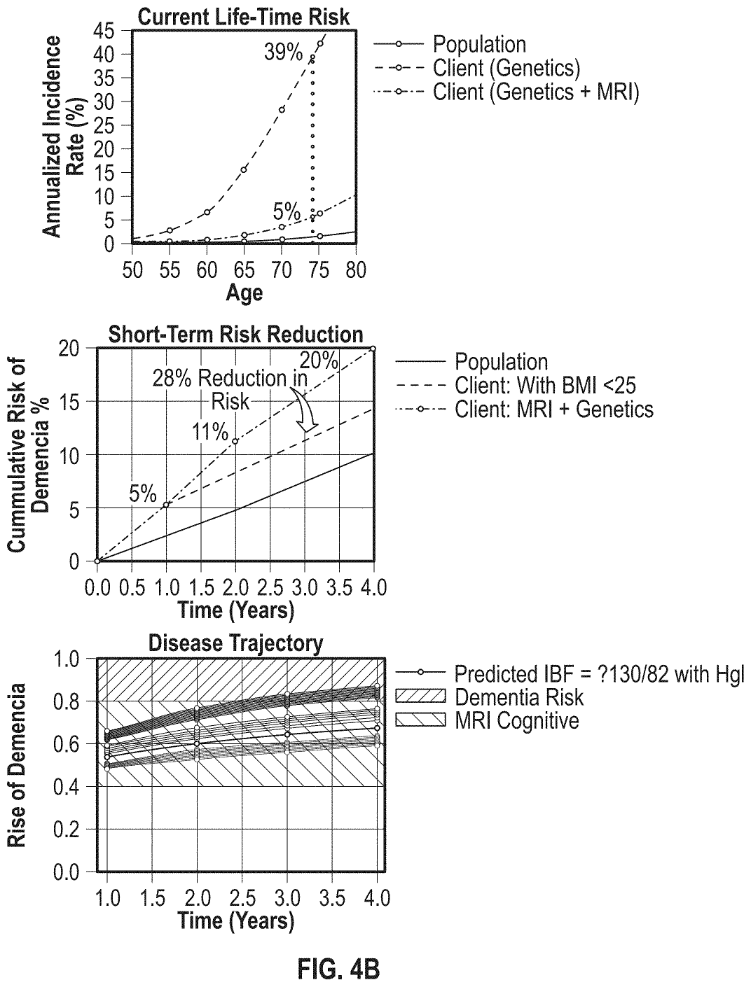

In some embodiments, the methods of the disclosure are used in determining short-term risk of developing dementia. Short-term risk usually evaluates the likelihood of developing dementia within four years, typically within three years, preferably within two years and especially within one year or less, e.g., six months. Utilizing an ensemble of boosted trees, a model was trained to predict whether or not an individual would develop dementia within a time frame: one, two, three, and four years. This technique was chosen because it provides both interpretability and performance. Next, the person's risk was calculated given in silico changes in modifiable risk factors. Cumulative short-term risk was then measured with in silico modification of actionable risk factors within one year of the baseline. To simulate the counter factual inference, it was assumed that brain morphology changes within a year are small. The decision tree identified that a threshold BMI of 25 was a marker, wherein BMI>25 increased risk for a subset of patients. Data is shown in FIG. 4.

Methods for Personalizing Risk Using Annualized Incidence Rate

In some embodiments, the methods of the disclosure are used in creating personalized life-time risk based on age, sex and other characteristics of an individual. A survival model framework is used to combine the probability of disease risk from the above described model with the population-based incidence rates from Global Burden of Disease per age bin from 55 years to 80+ years (Vos et al., Lancet, 390(10100):1211-1259, 2017).

As can be seen in the representative results presented in FIG. 4B, integration of genetic features with brain structure features (MRI data) drastically improves prognostic accuracy of the model. For instance, using a simple genetic model, an annualized incidence rate of developing dementia at age 74 is about 39% in subjects who are positive for the genetic feature. However, the annualized incidence rate in the population is much lower, at around 2%. Using a combination of genetic features and structural features, the annualized incidence rate is predicted to be much closer to, and thus better representative of, the actual incidence rate, at about 5%. That is, the overestimation of annualized incidence rate was reduced by nearly 7.5-fold (e.g., 19× with genetic data alone versus 2.5× with the combined genetic and MRI data) using a prediction model that utilizes the combination of genetic and structural features.

Methods for Determining Life-Time Risk

In some embodiments, the methods of the disclosure are used in determining life-time risk of being inflicted with dementia. Lifetime risk usually evaluates the likelihood of being afflicted with dementia for at least 5 years, at least 10 years, at least 15 years, at least 20 years, at least 25 years, at least 30 years, at least 40 years or more, e.g., at least 50 years, after undergoing diagnosis. A regularized linear regression model that combines both L1 and L2 penalties from the lasso and the ridge methods was used to select brain MRI features that were predictive of Alzheimer's disease compared to healthy normal. Using the selected MRI features and the polygenic risk score, a ridge regression model was built to predict the risk of Alzheimer's with age and gender as covariates.

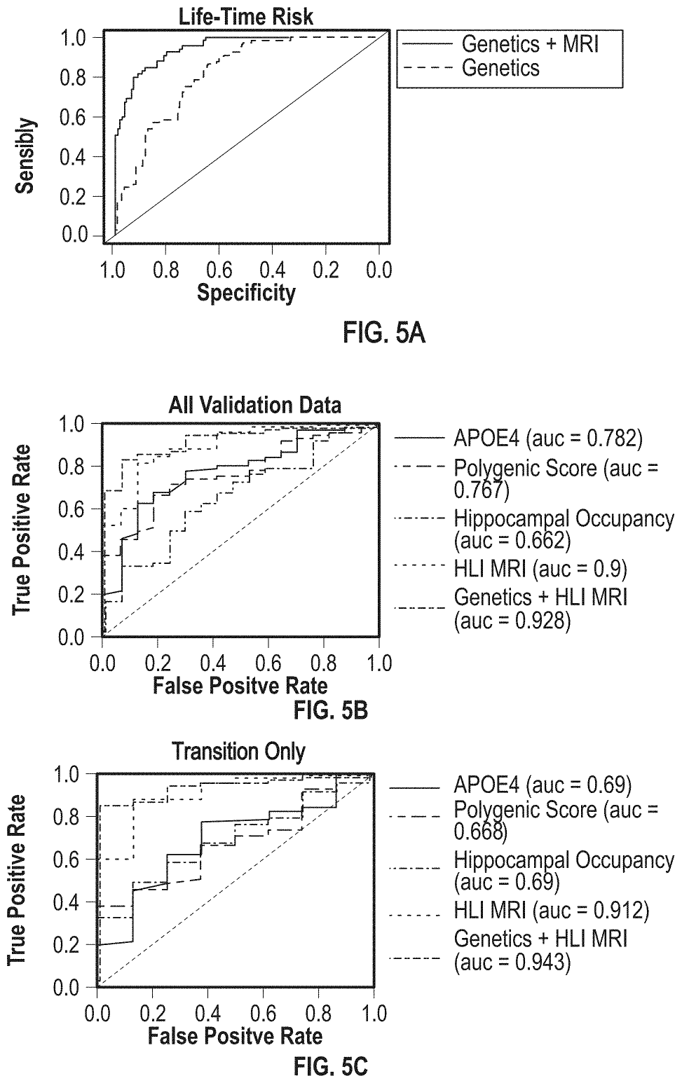

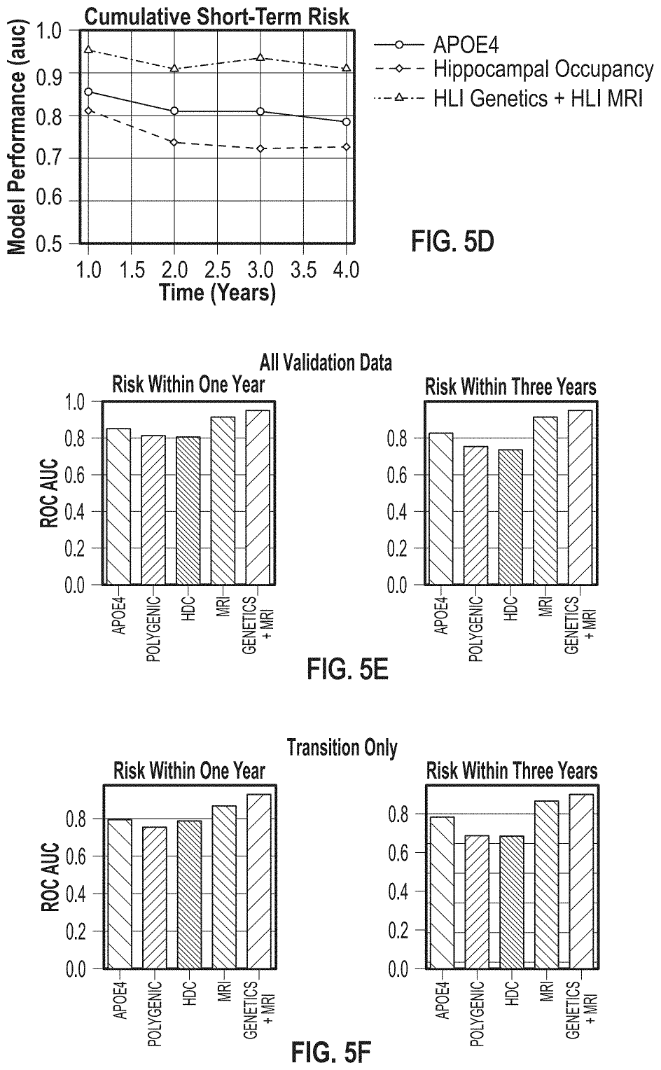

To evaluate the performance of the model, a validation data set can be used. Generally, the validation data set is separate from the training data set. The performance of the model can be assessed using Area Under Curve (AUC) of a receiver operating characteristic (ROC) curve. AUC is equal to the probability that a classifier will rank a randomly chosen positive instance higher than a randomly chosen negative one. Representative AUC curves are shown in FIG. 5A, wherein the AUC of the lifetime risk model was 0.96.

Methods for Determining Disease Trajectory

In some embodiments, the methods of the disclosure are used in determining disease progression trajectory via long short-term memory network. This model allows prediction of the rate, onset and severity of decline of memory with in silico modification of risk factors (BP, medication, dosage). For instance, the model can be used to predict the effect of blood pressure maintenance, medication, and other lifestyle changes on patterns and rate of memory loss.

The model is based on recurrent neural networks (RNNs) comprising, for instance, long short-term memory (LSTM). LSTM was chosen as it is widely utilized for sequence prediction, due to its ability to remember values over arbitrary time intervals while also incorporating new information. Disparate areas of machine learning have benefited from models that can take raw data with little preprocessing as input and learn rich representations of that raw data in order to perform well on a given prediction task. Accordingly, in some embodiments, the model incorporates a LSTM recurrent neural network and input dense layer for sequence prediction of the severity of cognitive decline.

In order to measure the predictive power of genetic markers and brain MRI features years prior to the onset of dementia, a Cox proportional hazards (CPH) model may be utilized. The model is a standard tool in survival analysis, used to identify the relationship between a set of variables, or risk factors, and the survival time (or, more generally, the time to an event of interest). The model aims to compute for each individual a hazard function, which describes how the risk of the onset of Alzheimer's evolves with time. The proportional hazards model assumes that the hazard function consists of two parts: a baseline hazard function, which is common to all the population, and a multiplicative factor, which is unique for each individual. A powerful property of the model is that it can incorporate “censored” samples; i.e., samples that left the study before the event of interest is observed.

Representative results, which are presented in FIG. 5B-5F, show that the representation-based learning outperforms the baseline using the multimodal input of the present disclosure. Accordingly, the systems and methods of the disclosure allow new ways for patient risk stratification based on a plurality of features (e.g., genetic features and brain structural features, optionally together with actionable features and/or epidemiological features).

Recommender

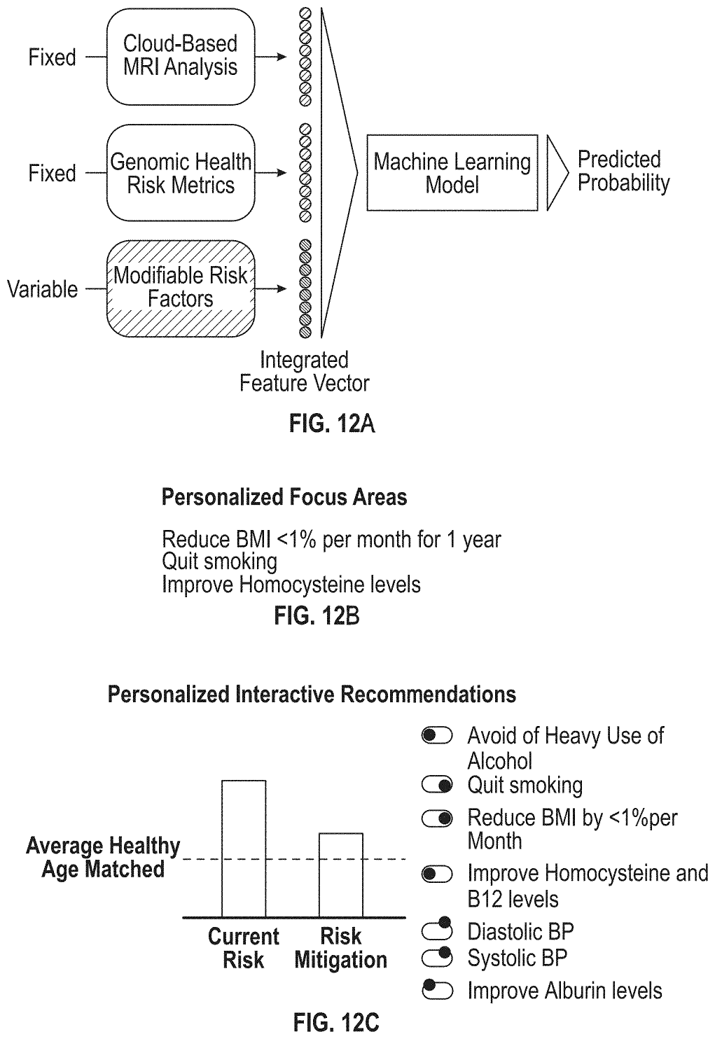

In certain embodiments, the disclosure relates to a recommender, which recommends certain actions for individuals at risk. Herein, given an individual's genetic markers and current brain structure and morphology, an individual's risk of cognitive decline in the short-term was re-calculated with in silico changes in modifiable risk factors (FIG. 12). The bounds on the variables are constrained with a priori knowledge of given medical literature and health guidelines (Table 5). In addition, the recommender is not allowed to recommend unachievable recommendations. For example, only <1% reduction in body mass per month is considered feasible.

TABLE 5
A priori knowledge to constrain recommender to only those
recommendations supported by medical literature.

| Risk Factor | Constraints |
|---|---|
| BMI | Towards healthy range, <1% weight loss per month |
| B12 | Can only increase |
| Homocysteine | Can only decrease |
| Albumin | Can only increase |
| Alcohol | Decrease only (metric is alcohol abuse) |
| Smoking | Can only recommend stopping smoking |
| Diastolic BP | Towards healthy range (60-80) |
| Systolic BP | Towards healthy range (100-120) |
| triglyceride levels | Can only decrease to healthy range |

The proposed changes go in to the one, two year, and three year model, where it is assumed that the changes take place one year from the baseline measurement.

The recommender can be used in two modes.

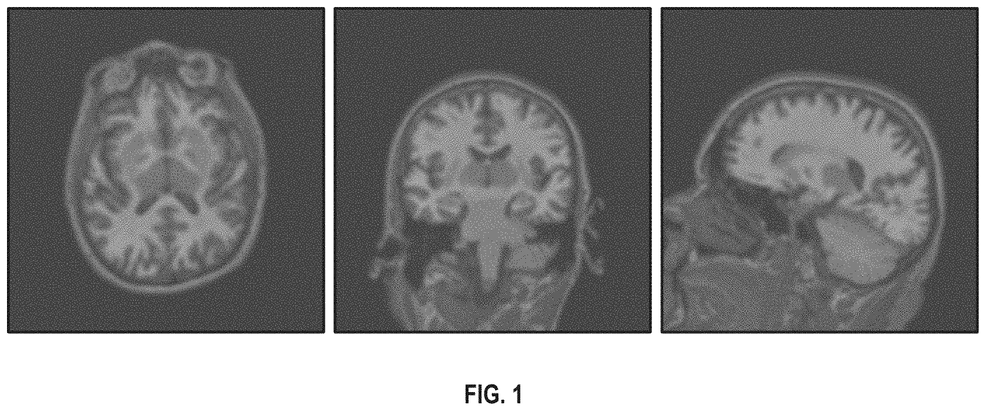

The first approach recalculates the risk for the individual for one, two, and three years given a proposed change such as reducing BMI to less than 25 as shown in FIG. 1B (middle panel). The result is shifted by one year giving the individual one year to make the proposed change.

The second approach proposes key focus areas and targets. The feature space is explored given a set of modifiable risk factors which are constrained by brain regions which are statistically associated with mild-cognitive impairment for the combination that minimizes the probability of decline. We leverage a bounded optimization and the Broyden-Fletcher-Goldfarb-Shannon (BFGS) algorithm to minimize the individual's risk with their current values for initialization.

For both modes, a proposed change given by either by a user or the optimizer is first evaluated to ensure it fulfills the constraints 2. For continuous features, the proposed value is calculated or evaluated based on the percentage change feasible within 1 year from the current value. The new variables are feed into one, two and three year models and a new probability of decline is calculated.

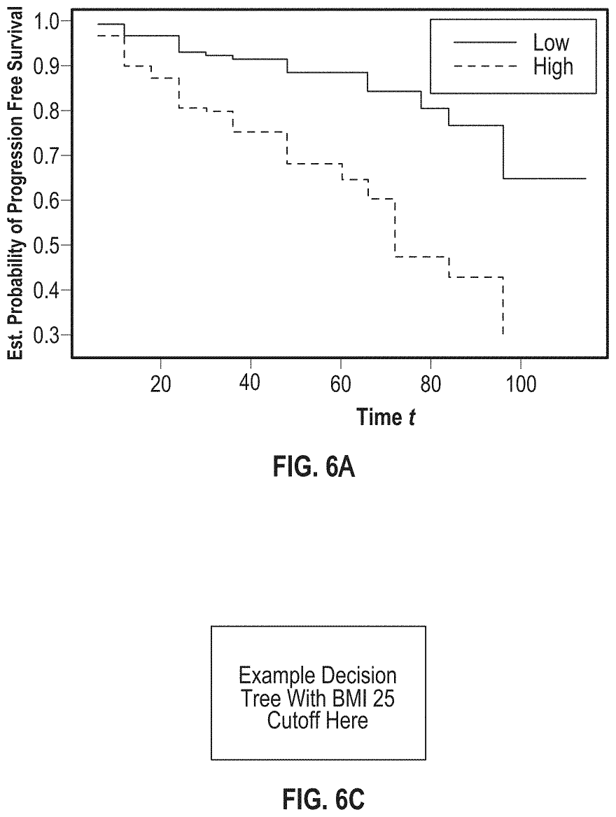

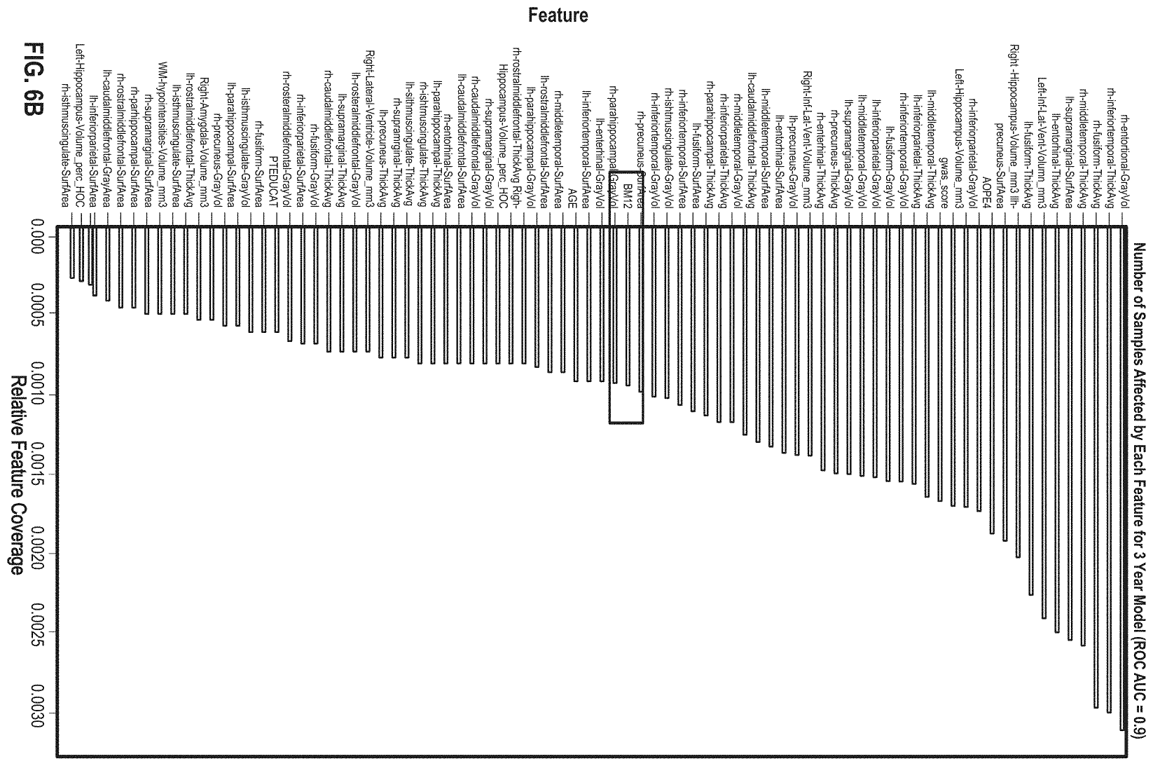

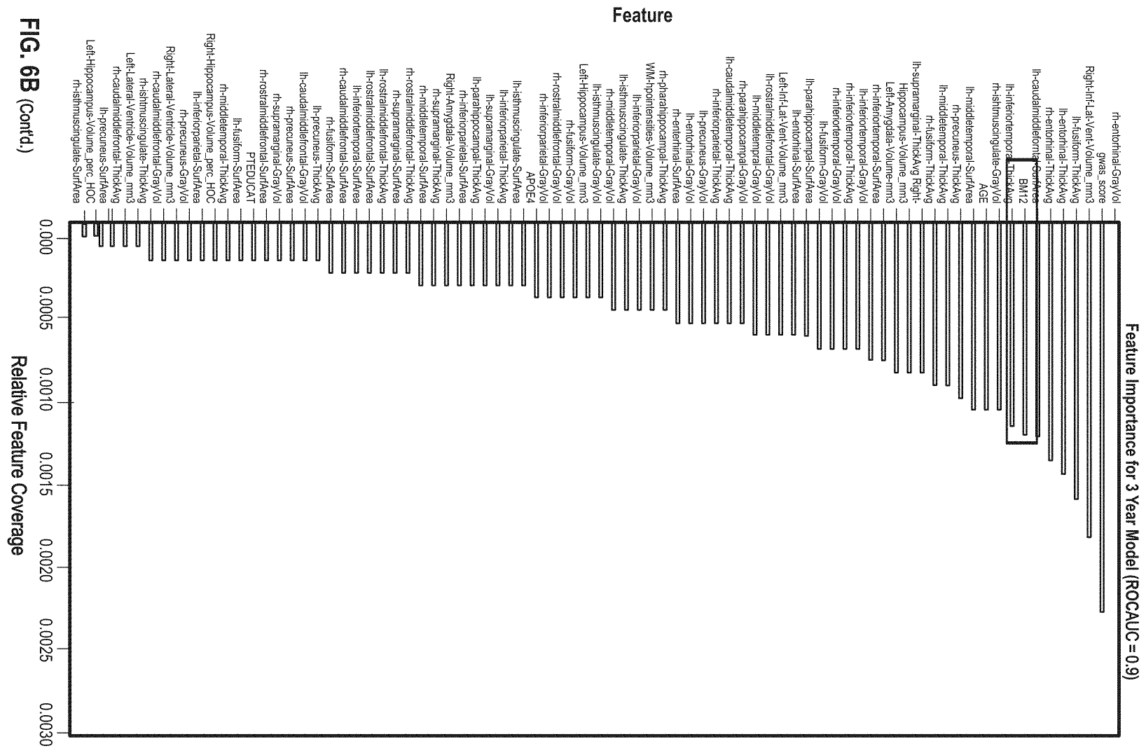

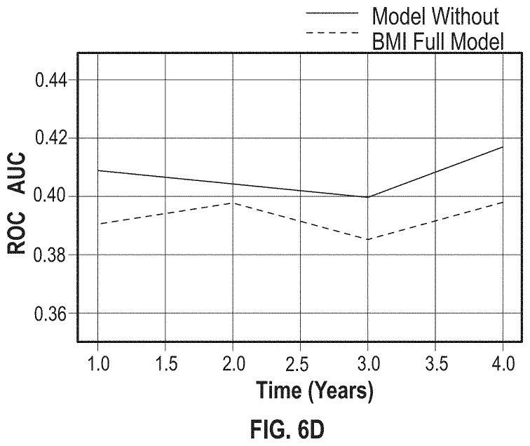

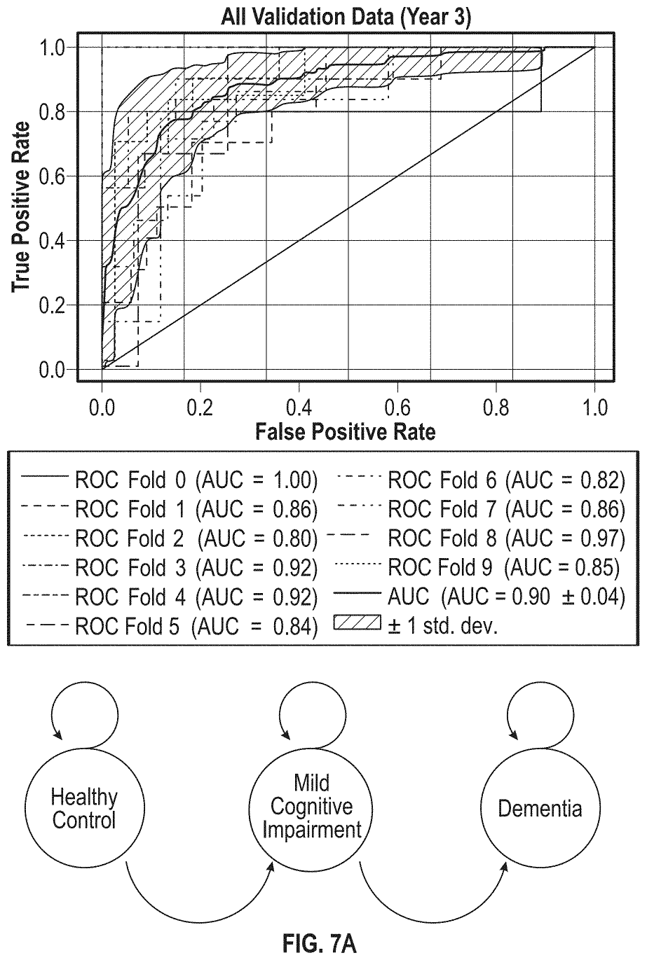

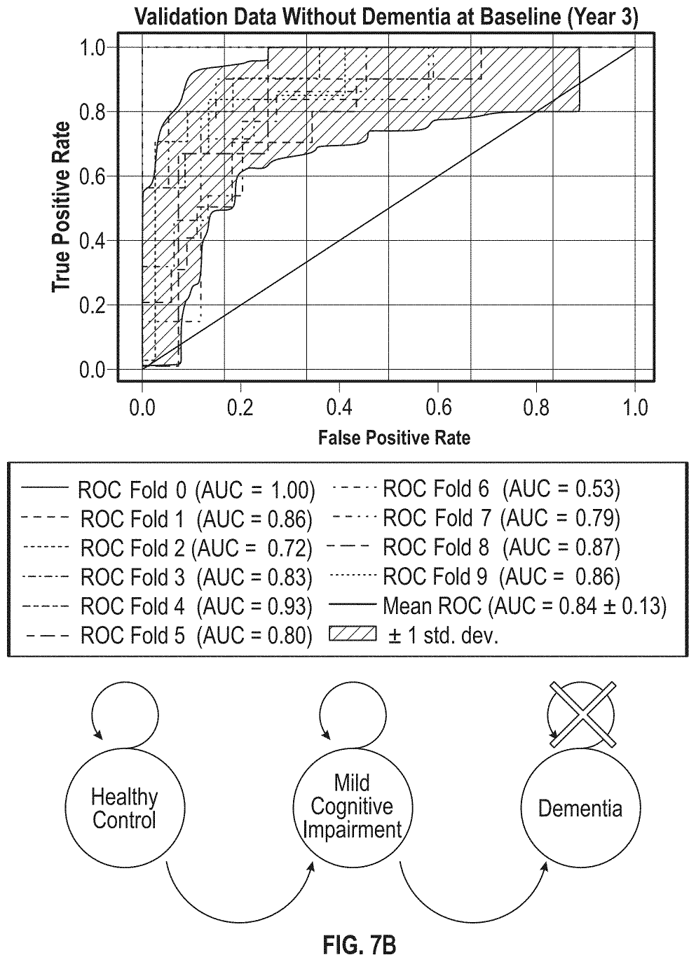

The use of action items per the recommender has measurable benefits. FIG. 6A-6C show in silico modification of actionable risk factors alters disease risk. FIG. 6A shows subtypes from multivariate survival model of disease progression shows that individuals with low, high, and normal BMI have statistically significant estimate of progression free survival. FIG. 6B shows feature importance and coverage for short-term risk model. FIG. 6C shows example of BMI inclusion in risk for in the ensemble of decision trees. Model learns AHA that BMI>25 increases risk for subset of individuals. FIG. 6D shows improvement of the model with the addition of actionable risk factors for both the short-term and long-term prognostication. The blue bars show MRI features of Table 4, in decreasing importance.

Short-Term Risk of Memory Decline

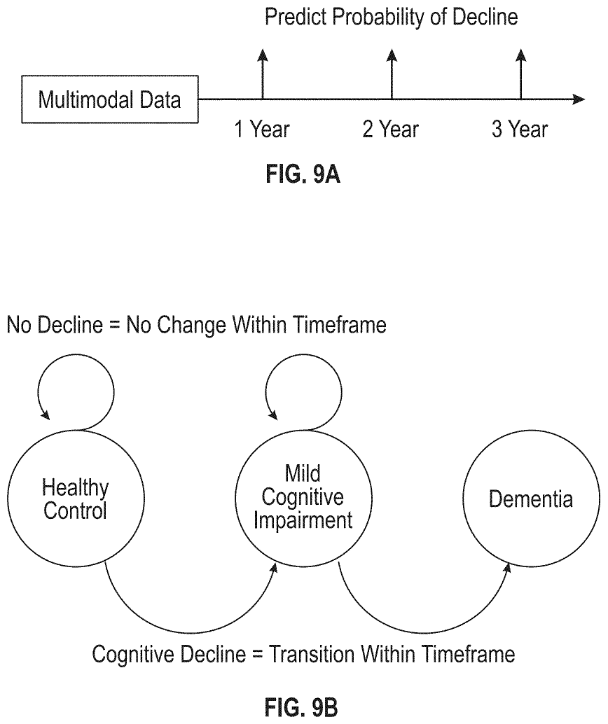

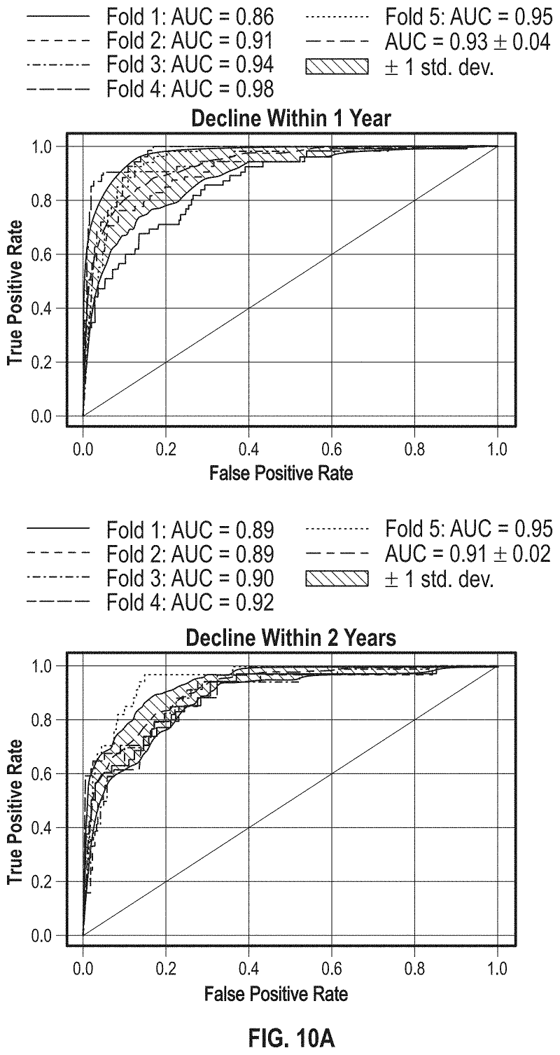

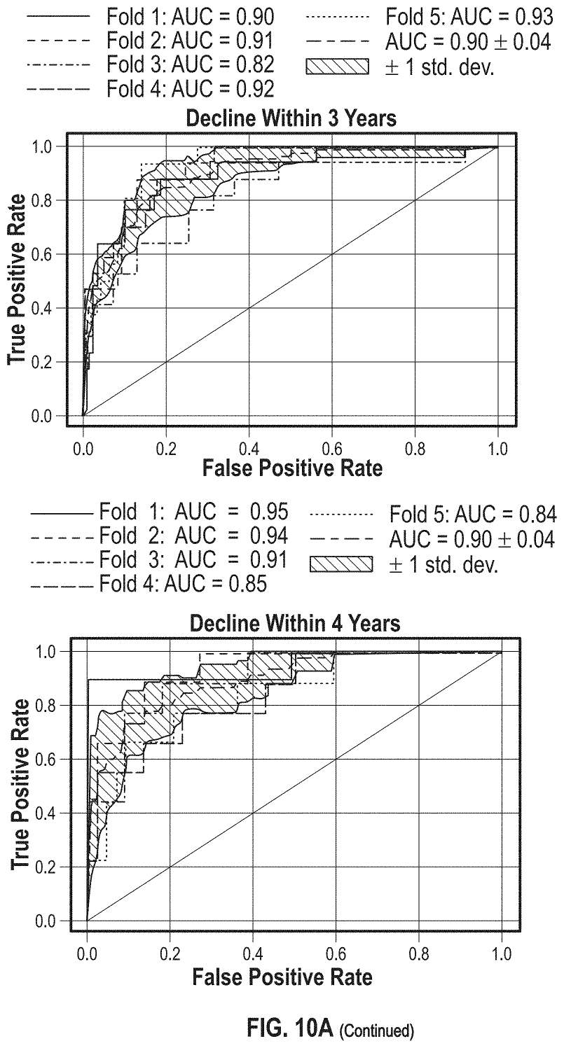

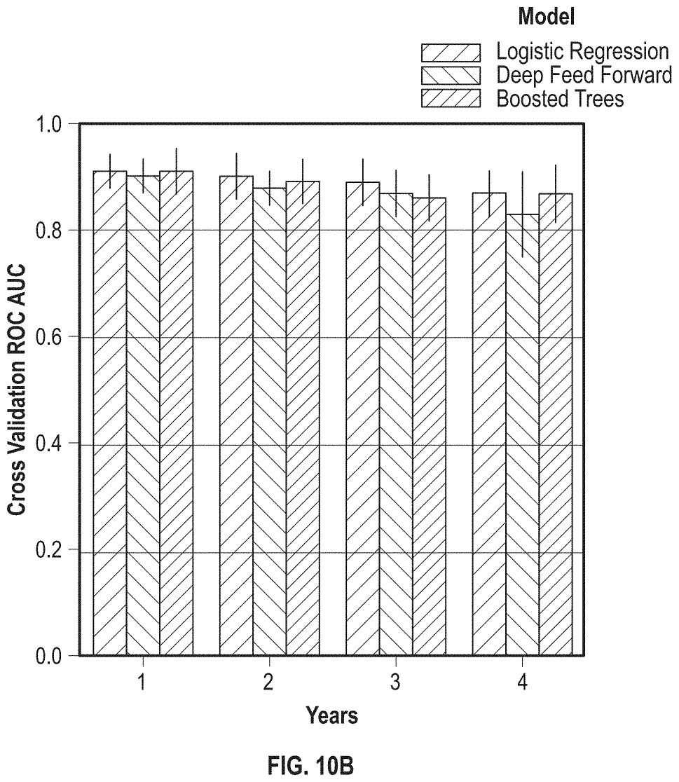

In some embodiments, the methods of the disclosure are used in determining short-term risk of memory decline. A set of binary classifiers were trained to predict whether or not an individual would have cognitive decline within a time frame: one, two, three, and four years. Cognitive decline was defined by a transition from normal to mild cognitive impairment (MCI) or progression from MCI to dementia (FIG. 9A). Various types of widely used modeling techniques were evaluated based on performance: including ensemble of boosted trees, deep feed forward networks, long-short term neural networks and logistic regression all widely used for classification tasks. We choose and ensemble of gradient boosted decision trees, where both interpretability and performance are desirable. Validation data are shown in FIG. 10.

The instant method can learn non-linear interactions between features, such that more personalized recommendations can be made, where certain factors are significant for sub-populations but not necessarily broadly applicable to the entire population. For example, individuals with a predisposition for vascular dementia, reducing BMI through diet and exercise would have a bigger impact on their risk.

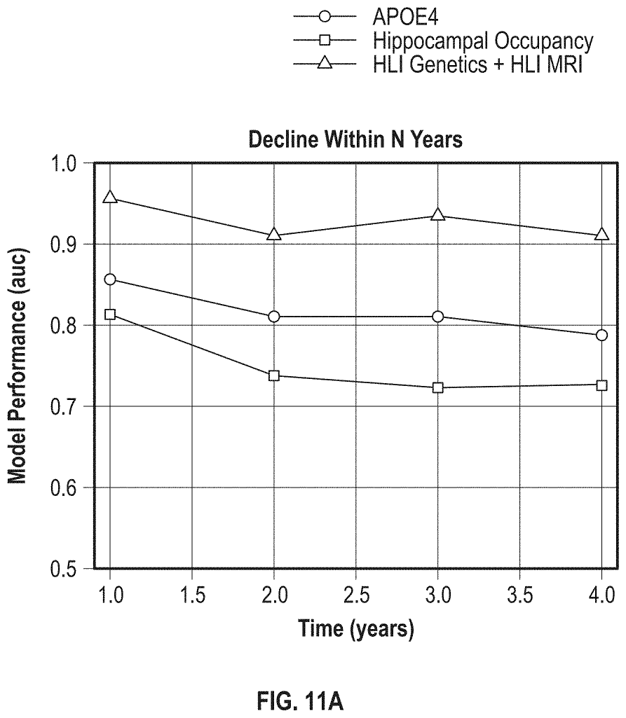

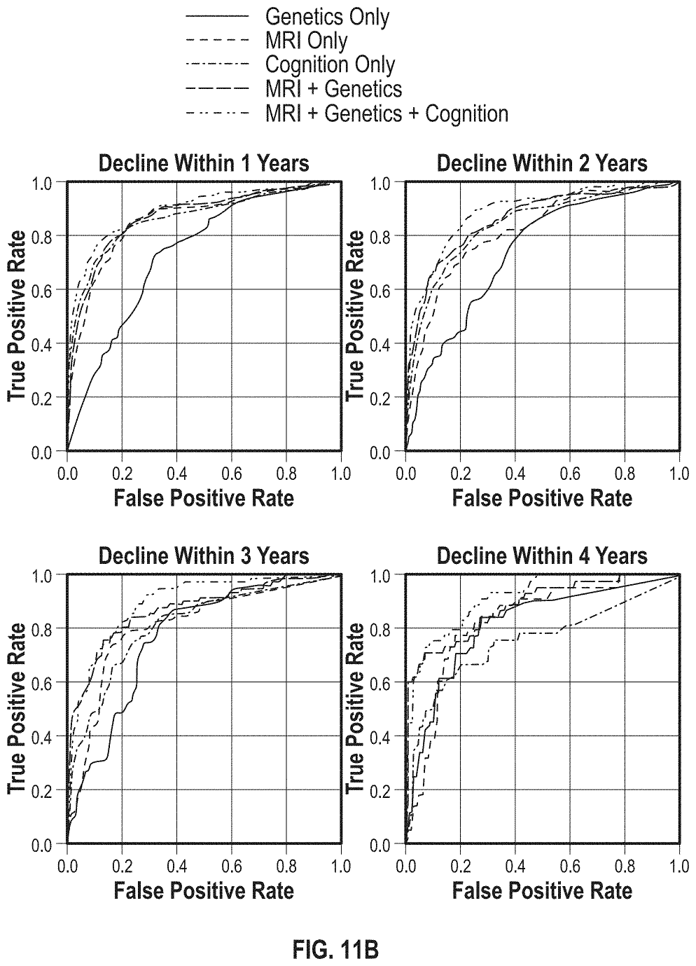

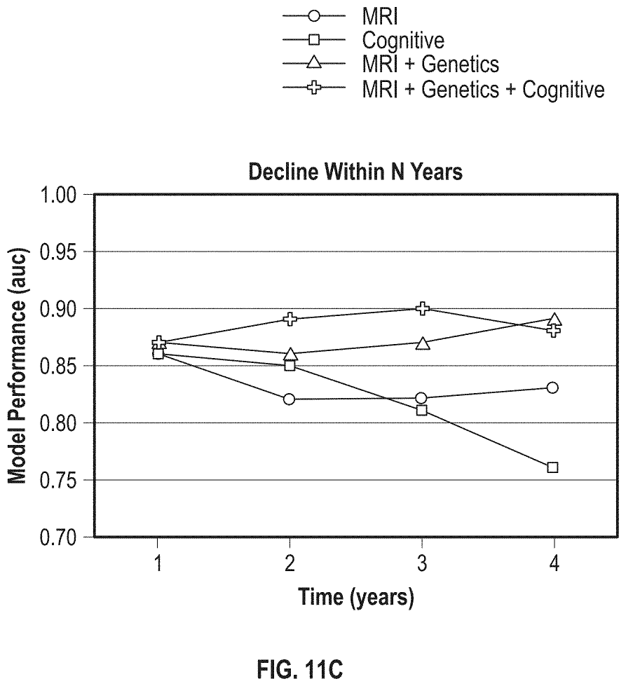

These models leverages MRI and genetics outperformed widely used biomarkers, specifically APOE4 status and Hippocampal Occupancy, in the prediction of cognitive decline within one year, two years, three and four years (FIG. 11A). Next we compared the predictive power of models with and without MRI features and cognitive tests for prediction of cognitive decline in the short term, were mean ROC AUCs from fivefold cross validation were evaluated. We observe that after 12 months models trained with MRI and GWAS always outperforms the models trained on MRI features, cognitive tests, or genetics markers only. This difference is accentuated the more time that has passed between the baseline measurement. Notably, the added value of a cognitive test to models with MRI and genetics is not significant three and four years post measurement, where MRI and genetics has similar performance. For FIG. 11B and FIG. 11C all hyperparameters were held constant for all years (e.g. learning rate, number of iterations, depth, gamma, lambda) to ensure a fair comparison, which results in a slightly reduced performance than the optimized MRI+genetics models and the MRI+genetics+cognitive models for each year. For the final model, the hyperparameters where tuned to get the optimal performance.

Workflow

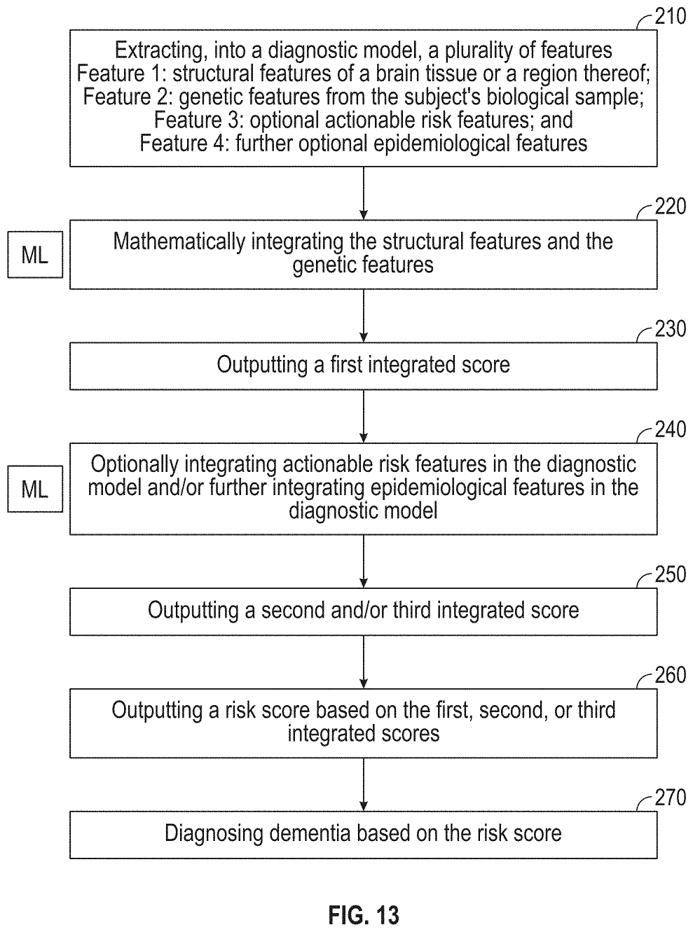

FIG. 13 shows a schematic diagram of the workflow of the disclosure and is used to diagnose dementia. There are many potential downstream applications to this technology, e.g., determining short-term or long-term risk; personalizing risk using annualized incidence rates; determining disease trajectory; identifying short-term risk of memory decline; and/or using a recommender.

In step 210 of method 200 of FIG. 13, a plurality of features is extracted. The features include (a) structural features of a brain tissue or a region thereof; and (b) genetic features from the subject's biological sample; optionally (c) actionable risk features; and further optionally (d) epidemiological features. These features may be received in appropriate files. For instance, genetic features may be received in a genetic data set (VCF or text file). Image features (e.g., MRI scans) may be received in pixel files (GIF, TIFF or any other format). Actionable risk features may be received in the form of binary tables (e.g., BMI>25?, 1 for yes; 0 for no). Epidemiological features may be received in appropriate datasets.

In step 220 of method 200 of FIG. 13, structural features and the genetic features are integrated. A machine learning algorithm may be used to integrate such discrete data.

In step 230 of method 200 of FIG. 13, a first integrated score is outputted.

In the optional step 240 of method 200 of FIG. 13, actionable risk features are integrated in the diagnostic model and/or further optionally epidemiological features are integrated in the diagnostic model. Again, machine learning algorithms may be used to integrate such discrete data pertaining to actionable risk features and/or epidemiological features.

If the optional step 240 of method 200 of FIG. 13, is implemented, then in step 250, a second score and/or third integrated score is outputted.

In step 260 of method 200 of FIG. 13, a risk score based on the first, second, or third integrated scores is outputted. A variety of different measures of association is routinely used in epidemiology. The most common are relative risk (RR; risk ratio) and odds ratio (OR). Risk ratio is often used in cohort studies and may be defined as the relative risk associated with a risk factor, e.g., RR=R1/R0, where R1 is the rate in an exposed group versus RO, the rate in a non-exposed group. RR is thus a risk multiplier on top of a baseline risk RO, where the segment of the RR above 1 represents elevation in risk. Thus, a RR of 1.0 or greater indicates an increased risk, a RR of less than 1.0 indicates decreased risk, and a RR of 2 represents a 100% increase in risk. OR is an epidemiological measure of association expressing disease frequency in terms of odds, and is defined as the odds of disease in the exposed population divided by the odds of disease in the unexposed population. OR is more often used in case-controlled studies, and may involve a comparison of disease cases with the prevalence among non-cases for controls. Both RR and OR characterize the association between the exposure and the disease in relative terms, and both reflect the frequency of disease occurrence among exposed subjects as a multiple of the rate among unexposed subjects.

In step 270 of method 200 of FIG. 13, dementia is diagnosed based on the risk score. In various embodiments, a subject is diagnosed with dementia if the subject's score exceeds a pre-set risk score threshold. In various embodiments, the pre-set risk score threshold is set based on the subject's demographic information (e.g., age, ethnicity, socioeconomic strata, place of residence, etc.). In various embodiments, the pre-set threshold is set based on the subject's family medical history.

Generally, a machine learning approach may be incorporated to systemically integrate various features. The approach may be applied at any step of the method, although it may be advantageous to implement the machine learning at step 220. If optional features such as actionable risk features and/or epidemiological features are utilized, then machine learning may be implemented at these optional step(s) 240 as well. In this regard, in the purely illustrative method of FIG. 13, a machine learning (ML) algorithm is applied at step 220 and/or optionally at step 240 to build the model. The ML algorithm may comprise employing a deep learning algorithm such as, e.g., using neural networks, with applicable training data sets and specific weighthing factors optimized by backpropogation, to analyze interrelationships between discrete features such as image data and/or genetic data and deduce the functional significance thereof.

In some embodiments, the ML is trained with an in silico dataset. For example, the in silico dataset may include GWAS data (e.g., genetic features associated with dementia). The ML algorithm may also be trained with phenotypic MRI data, e.g., MRI of subjects with or without dementia; preferably, subjects with Alzheimer's disease. Next, the genetic features and the image features are concatenated using mathematical algorithms and an integrated score is outputted.

The architecture of the machine learning approach will be discussed in greater detail below.

Machine Learning (ML)

Not being bound to a single embodiment and purely for the purpose of illustration, a machine-learning algorithm was integrated into the existing methodology at an individual, or combination of individual steps, in accordance with various embodiments herein. ML can be incorporated to optimize the results coming out of the algorithm (e.g., neural network, ML algorithm, etc.), by utilization of inputted training data sets, cross reference of output to known answers, backpropagation, and adjustment of weighting factors and parameters associated with the given ML algorithm in a repeating loop to arrive at a threshold quality of data output. In subsequent steps, the prediction power of the model on the test dataset may be validated, e.g., using a probability model such as logistic regression (e.g., optimized or trained in conjunction or in the alternative). Optionally, a resampling may be performed to obtain an unbiased appraisal of the model's likely future performance. Features of ROC curve, such as, area-under-the curve (also called c-index) or concordance probability from a statistical test such as the Wilcoxon-Mann-Whitney test, may provide a good summary measure of pure predictive discrimination.

### Computer-Implemented Systems

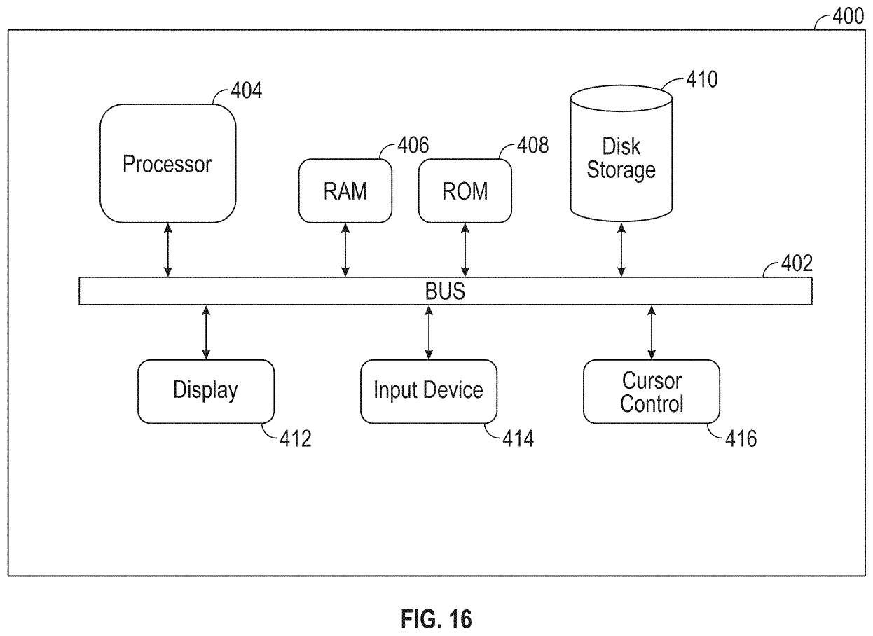

FIG. 16 is a block diagram that illustrates a computer system 400, upon which embodiments of the present teachings may be implemented. In various embodiments of the present teachings, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (i.e., x) and a second axis (i.e., y), that allows the device to specify positions in a plane. However, it should be understood that input devices 414 allowing for 3 dimensional (x, y and z) cursor movement are also contemplated herein.

Consistent with certain implementations of the present teachings, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400 of FIG. 16, whereby processor 404 would execute the analyses and determinations provided by these engines, subject to instructions provided by any one of, or a combination of, memory components 406/4008/410 and user input provided via input device 414.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical, FLASH memory and non-optical data storage devices. The computer readable medium can also be distributed over a network coupled computer systems so that the computer readable code is stored and executed in a distributed fashion.

Systems

The disclosure relates to systems for diagnosing dementia comprising a receiver for receiving a plurality of features comprising (1) structural features of a brain tissue of the subject or a region thereof; (2) genetic features from the subject's biological sample; (3) optionally actionable risk features; and (4) further optionally epidemiological features; a first integrator for integrating structural features and genetic features to output a first integrated score; an optional second integrator for integrating actionable risk features in the diagnostic model to output a second integrated score and a further optional third integrator for integrating the epidemiological features in the diagnostic model to output a third integrated score; and a scorer for determining a risk (i.e., risk score) of dementia based on the first, second or third integrated score, wherein the risk score is used to diagnose dementia. In various embodiments, a subject is diagnosed with dementia if the subject's score exceeds a pre-set risk score threshold. In various embodiments, the pre-set risk score threshold is set based on the subject's demographic information (e.g., age, ethnicity, socioeconomic strata, place of residence, etc.). In various embodiments, the pre-set threshold is set based on the subject's family medical history.

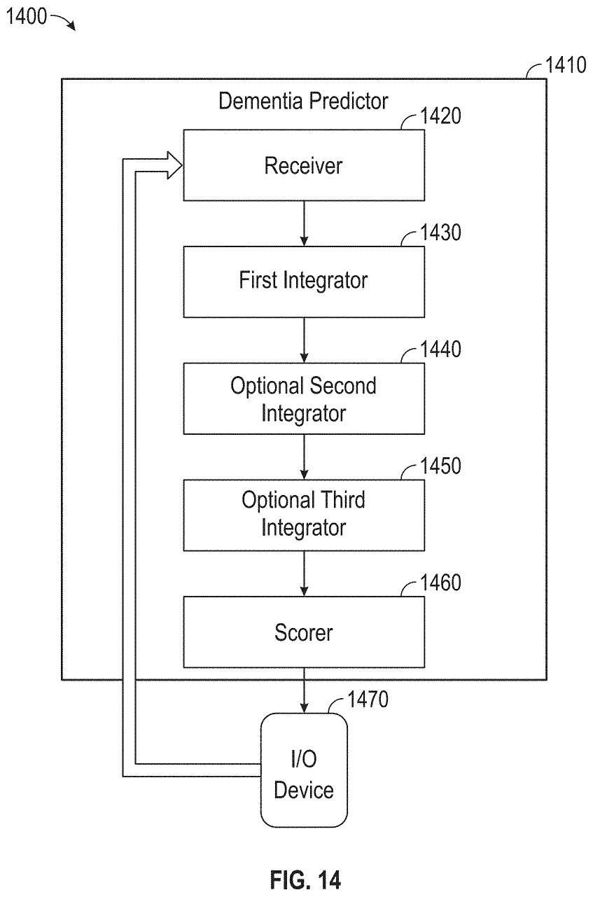

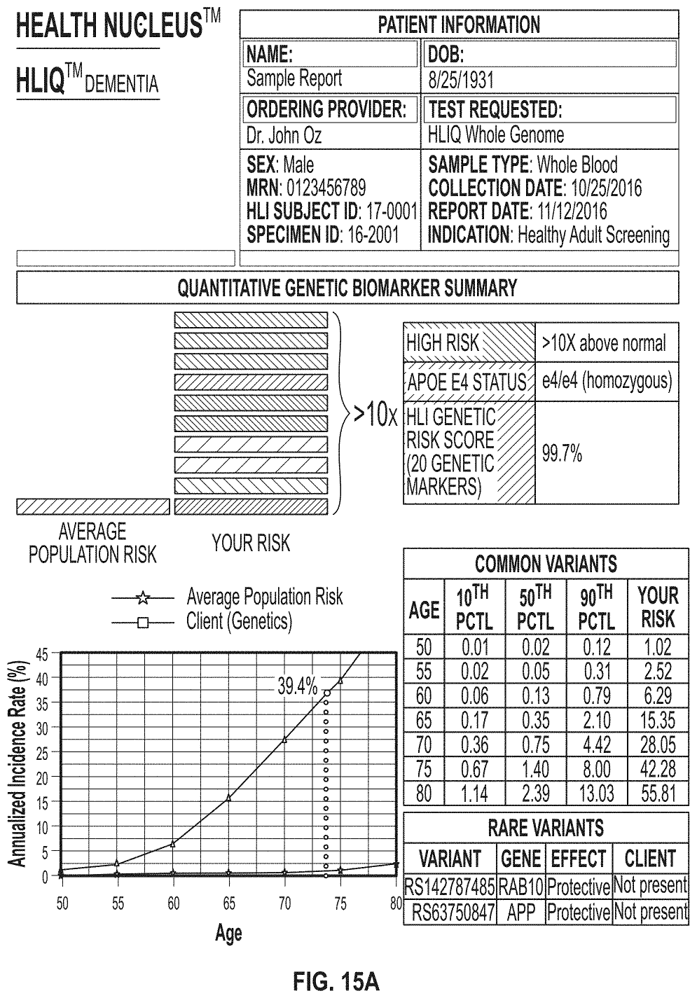

FIG. 14 shows a schematic diagram of a representative system 1400 of the disclosure. Specifically, a representative Dementia Predictor 1810 is shown, which is useful for diagnosing dementia. Dementia Predictor 1810 comprises three modules and can be communicatively connected to an input/output device (I/O device). A first module, Receiver 1420 contains components and/or software for receiving datasets of features, e.g., structural features of a brain tissue of the subject or a region thereof and genetic features from the subject's biological sample, optionally together with actionable risk features and/or epidemiological features. It should be noted that owing partly to the different types of data that is inputted (e.g., text file of genetic information and image file of MRI data), different types of receivers may be implemented (e.g., a gene sequencer and an MRI scanner). The Receiver 1420 is communicatively connected to a second module, the First Integrator 1430. First Integrator 1430 contains components and/or software for integrating the structural features (e.g., brain phenotype data based on MRI) and the genetic features (e.g., SNP data based on WGS or NGS). First Integrator 1430 may be communicatively connected to Second Integrator 1440 and/or Third Integrator 1450. The optional second integrator integrates actionable risk features in the diagnostic model to output a second integrated score and the further optional third integrator integrates epidemiological features in the diagnostic model to output a third integrated score. If the optional Second and Third Integrators are absent, the first integrator is directly and communicatively connected to a third module, the Scorer 1460. However, if the optional Second Integrator 1440 and/or Third Integrator 1450 are included, then Scorer 1460 is communicatively connected with these downstream integrative components. Scorer 1460 contains components and/or software for determining a risk of dementia based on the first, second or third integrated score. Scoring module 1840 is communicatively connected to an input/output (I/O) device, e.g., a server or a computer or a smartphone, which in turn may be connected to the Dementia Predictor 1810. Ideally, the I/O device has a display, wherein the output, i.e., whether the protein of interest or the binding pocket therein is intolerant to variation, is displayed.

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1

Background: Accurate prediction of dementia at the individual level may enable healthcare providers to provide a more personalized approach to treating dementia. A study was conducted, incorporating various risk factors to build a multimodel that allows for personalized diagnosis of every subject and further recommend action items that can be implemented or incorporated to reduce the individual's risk of onset, location, duration, character, progression, intensity/severity, or timing of dementia or a symptom related thereto (e.g., stress reduction, B12 supplementation, weight loss, alteration of medication regimen). The results of these multimodal systems and/or methods may be used to not only identify or group at-risk subjects, but also allow clinicians to make appropriate recommendations on prophylaxis or therapy of dementia (e.g., via drug therapy or lifestyle changes)

Methods

I. Feature Extraction

For each dataset the following features were extracted:

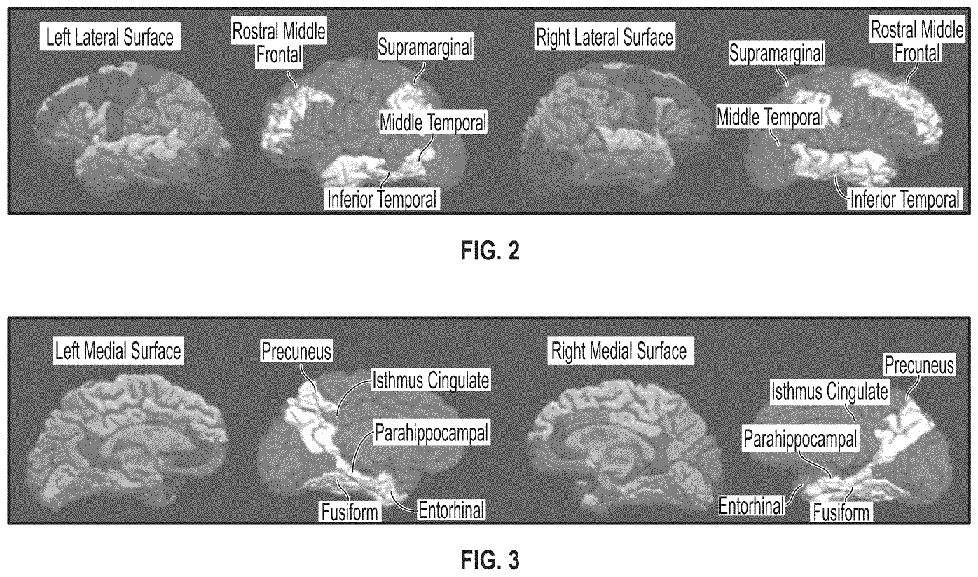

Structural MRI: Feature extraction was performed with the Freesurfer image analysis suite, which is freely available for download online (on the world-wide-web at surfer(dot)nmr(dot)mgh(dot)harvard(dot)edu/). The processing includes removal of non-brain tissue, automated segmentation of subcortical structures, cortical surface reconstruction, and cortical parcellation. Calculated features include volume, cortical thickness, and cortical surface area. Seventy-seven features, including cortical thicknesses, surface areas, volumes were extracted for regions known to have an effect size greater than 1 from Karow et al. (Radiology, 256(3): 932-942, 2010). See the representations shown in FIG. 1-FIG. 3.

Risk factors: Labs, medications, and vital signs that had corresponding mitigating actions were included into the models and evaluated for significance.

Genetics: A polygenic risk score was calculated using twenty known genetic markers associated with Alzheimer's disease from a published GWAS study. The score was calculated by summation of the number of risk alleles carried by an individual for each variant, weighted by the effect size (log2(Odds ratio)) from the GWAS. The score for each individual was normalized to a reference population of matching ancestry to account of any allele frequency differences between ancestral populations.

A schematic outline of the methods of the disclosure is provided in FIG. 4A.

Short-Term Risk

An ensemble of boosted trees was trained to predict whether or not an individual would develop dementia within a time frame: one, two, three, and four years. This technique was chosen because it provides both interpretability and performance.

Next, the person's risk was calculated given in silico changes in modifiable risk factors.

Cumulative short-term risk with in silico modification of actionable risk factors within one year of the baseline. To simulate the counter factual inference, we assume that brain morphology changes within a year are small. The decision trees learned that a cut off of BMI=25 increases risk for a subset of patients, which is consistent with the AHA recommendations where a BMI of >25 is considered overweight. Results are presented in FIG. 4B.

Assessment of Life-Time Risk of Dementia

A regularized linear regression model combining both L1 and L2 penalties from the lasso and the ridge methods was used to select brain MRI features that were predictive of Alzheimer's disease compared to healthy normal. Using the selected MRI features and the polygenic risk score, a ridge regression model to predict the risk of Alzheimer's was built with age and gender as covariates. To evaluate the performance of the model, we used a validation data set, which was separate from the training data set. The performance of the model was measured using Area Under Curve (AUC) of a receiver operating characteristic (ROC) curve (FIG. 5A). AUC is equal to the probability that a classifier will rank a randomly chosen positive instance higher than a randomly chosen negative one. The AUC of the lifetime risk model was 0.96.

To create personalized lifetime risk based on age and sex of an individual, we used a survival model framework to combine the probability of disease risk from the above-described model with the population-based incidence rates adjusted for mortality by other factors from Global Burden of Disease per age bin from 55 years to 95+ years. Data, which are presented in FIG. 5A, show Receiver Operator Curves (ROC) for personalized lifetime risk with a regularized generalized linear model with Elastic net for feature selection. It can be seen that integration of imaging features with the genetic features greatly improves ROC compared to genetic features alone.

Disease Trajectory

Disease progression trajectory via long short-term memory network for the prediction of the rate, onset and severity of decline with in silico modification of risk factors (BP, medication, dosage). LSTM was chosen as it is widely utilized for sequence prediction, due to its ability to remember values over arbitrary time intervals while also incorporating new information. In addition, recurrent neural networks are known to have performed well with rare events in sequences. Disparate areas of machine learning have benefited from models that can take raw data with little preprocessing as input and learn rich representations of that raw data in order to perform well on a given prediction task. In particular, we trained a LSTM recurrent neural network and input dense layer for sequence prediction of the severity of cognitive decline. We compared the neural network to a cox survival analysis, which was also used to understand the importance of the modified risk factors due to its interpretability. We find that the representation-based learning outperforms the baseline given our unique multimodal input. We believe that this method suggests a new avenue for patient risk stratification.

### Example 2: Use of Cox Proportional Hazard Ratios to Assess Risk

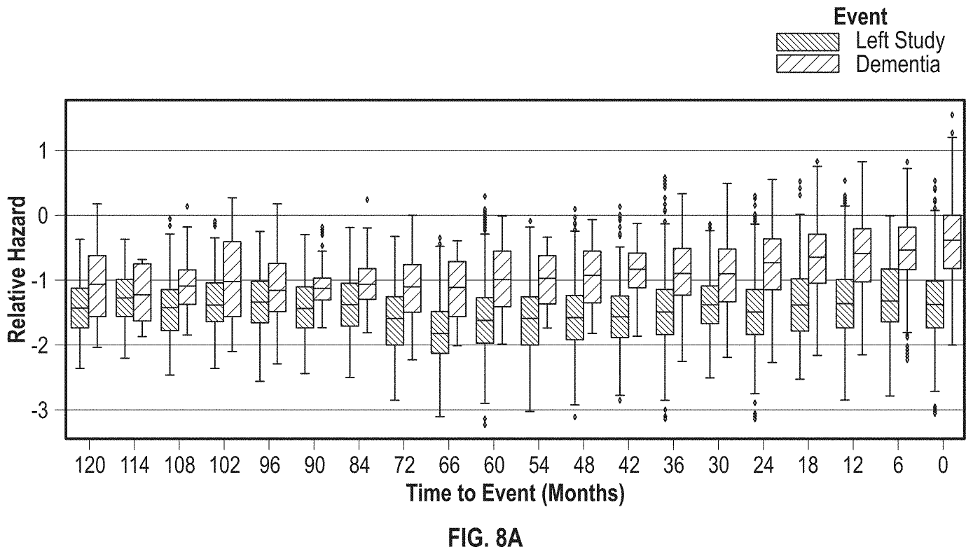

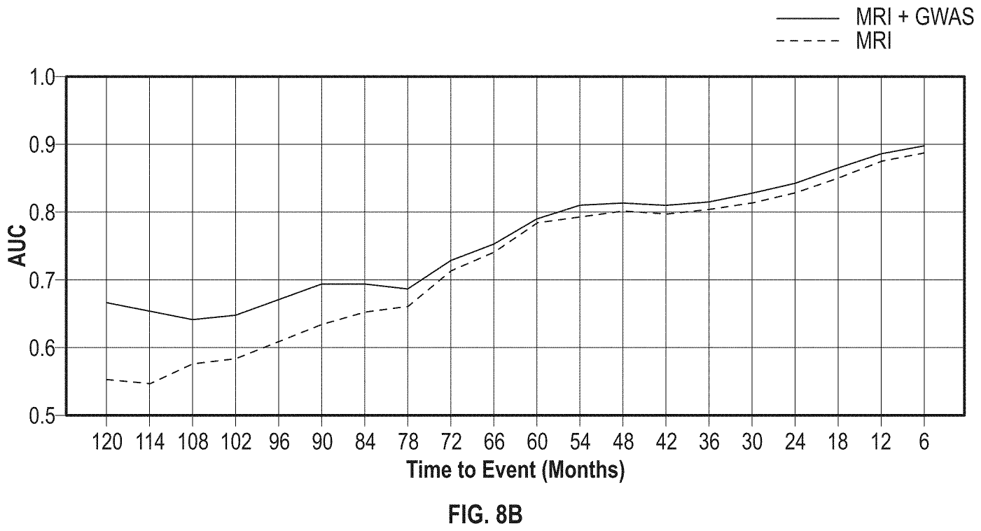

In order to measure the predictive power of genetic markers and brain MRI features years prior to the onset of dementia, we utilized a Cox proportional hazards (CPH) model. The model is a standard tool in survival analysis, used to identify the relationship between a set of variables, or risk factors, and the survival time (or, more generally, the time to an event of interest). The model aims to compute for each individual a hazard function, which describes how the risk of the onset of Alzheimer's evolves with time. The proportional hazards model assumes that the hazard function consists of two parts: a baseline hazard function, which is common to all the population, and a multiplicative factor, which is unique for each individual. A powerful property of the model is that it can incorporate “censored” samples; i.e., samples that left the study before the event of interest is observed. Results are shown in FIG. 8.

In FIG. 8A, we analyze the hazard score for individuals that have onset of dementia versus those who do not (i.e., they leave the study without ever transitioning). The results show that the closer you are to the onset of dementia, the more predictive the score is. In FIG. 8B, this is quantified in terms of the AUC for the task of discriminating individuals that transition to dementia in t months versus those that remain at least t months in the study without transitioning. We observe that the CPH model trained on MRI and GWAS always outperforms the model trained on MRI features only, and this difference is accentuated the farther away we are from the time of onset.

### Example 3: Clinical Assessment

Measurement of Genetic Markers

Blood specimens are collected and genomic DNA extraction is carried out using a standard kit following manufacturer's recommendations. DNA is eluted in 50 uL Elution Buffer (EB, Qiagen) and stored at 4° C. until used. Double-stranded DNA is quantified with a Quant-iT fluorescence assay (Life Technologies). The genomic DNA is normalized and sheared with a Covaris LE220 instrument. Next Generation Sequencing (NGS) library preparation is carried out using the TruSeq Nano DNA HT kit (Illumina Inc), essentially following manufacturer's recommendations. Alternately, next whole genome sequencing (WGS) may be carried out using standard methods. Individual DNA libraries are characterized in regards to size and concentration using a LabChip DX One Touch (Perkin Elmer) and Quant-iT (Life Technologies), respectively. Libraries is normalized to 2-3.5 nM and stored at −20° C. until used.

The clustering and sequencing may be carried out using an Illumina HiSeqX sequencer utilizing a 150 base paired-end single index read format.

For read mapping and/or genotyping of sequenced data, the following protocol may be implemented: base call (BCL) files are used to map reads to a human reference sequence (hg38 build) using ISIS Analysis Software (v. 2.5.26.13; Illumina). The hg38 reference sequence was modified by masking the pseudoautosomal region of chrY. The ISIS Isaac Aligner (v. 1.14.02.06) identifies and marks duplicate reads, which are removed from downstream analysis. The resulting bam files are characterized using Picard (v. 1.113-1.131), and input to the ISIS Isaac Variant Caller (v. 2.0.17). The Isaac Variant Caller is used with default settings, and yielded genomic VCF files (gVCF). For computation of accuracy, single nucleotide variants with a “PASS” flag is compared to GIAB (v. 2.19). The data for the GiaB high confidence region are derived from 11 technologies: BioNano Genomics, Complete 3 Genomics paired-end and Long Fragment Read, Ion Proton, Oxford Nanopore, Pacific Biosciences, SOLiD, 10× Genomics GemCode™ WGS, and Illumina paired-end, mate-pair, and synthetic long reads.

For validation, a plurality of samples may be tested. Unique samples representing the wild-type genotype are tested for heterozygous variant(s). First, common variants (>0.1 percent variant frequency in the relevant population) are tested with a plurality of unique samples. Rare variants (<=0.1% variant frequency in the relevant population) may be tested with at least three unique samples. To test samples that are homozygous for the reported variant(s), variants with >2% variant frequency in a relevant population may be tested with about 20 unique samples. Variants with a frequency in the relevant population <2% and >0.5% may be tested with about 10 unique samples. Variants with a frequency in the relevant population <0.5% must be tested with at least three unique samples. If variants with a frequency of <0.5% are not found within the relevant population and homozygous samples are not tested, then the test results may be omitted.

Image data: Three-dimensional T1-weighted magnetic resonance (MR) images from either 1.5T OR 3T MR imaging units are used. Preferably, standard methodologies, which produce very similar spatial resolution, contrast, and SNR properties, across vendors and across various systems within each vendor product line, are implemented.

For individual scan, localizer/scout scan or straight sagittal 3D scan may be implemented. The sagittal scan includes T1-weighted sequence such as magnetization-prepared 180 degrees radio-frequency pulses and rapid gradient-echo (MP-RAGE) or equivalent. For phantom—quality control scans, localizer/scout scan and/or straight sagittal 3D MP-RAGE may be implemented.

The details on MRI imaging, including, software used in the capture of images can be found in the Alzheimer's Disease Neuroimaging Initiative (ADNI) MRI Technical Procedures Manual, available on the web at adni(dot)loni(dot)usc(dot)edu/wp-content/uploads/2010/09/ADNI_MRI_Methods_Non-ADNI_Studies.pdf (version 1: dated Jun. 26, 2006), which disclosure is incorporated by reference herein its entirety.

For image feature extraction, Freesurfer image analysis suite (available via the web at surfer(dot)nmr(dot)mgh(dot)harvard(dot)edu) or equivalent software may be used. The processing includes removal of non-brain tissue, automated segmentation of subcortical structures, cortical surface reconstruction, and cortical parcellation. Calculated features include volume, cortical thickness, and cortical surface area. Seventy-nine features, including cortical thicknesses, cortical surface areas, and volumes were extracted for regions known to show atrophy in Alzheimer's disease (Table 4). Age matched normative percentiles were also created. Data was normalized to intracranial volume and the hippocampal occupancy was calculated.

Additional risk factors and demographics: Optionally, additional risk factors may be implemented in the calculation, which may be applied selectively in some models. For instance, a first model may evaluate age adjusted lifetime risk of dementia; a second model may evaluate short-term risk of cognitive decline; and a third model may evaluate actionable recommendations for short-term risk of cognitive decline. Some risk factors may be included in all models; whilst other risk factors are specific to a model. Table 6 lists some additional factors that may be included in the model.

TABLE 6
Additional factors included in the three models.

| RISK FACTORS & | |
|---|---|
| DEMOGRAPHICS | UTILIZED IN MODELS |
| Age | All models |
| Gender | All models |
| AVLT | Optional addition for Model 2&3 |
| MMSE | Optional addition for Model 2&3 |
| B12 | Model 3 only |
| Triglycerides | Model 3 only |
| BMI | Model 3 only |
| B12 | Model 3 only |
| Homocysteine | Model 3 only |
| Albumin | Model 3 only |
| Alcohol | Model 3 only |
| Smoking | Model 3 only |
| Diastolic BP | Model 3 only |
| Systolic BP | Model 3 only |

From the foregoing description, one skilled in the art can easily ascertain the essential characteristics of the methods and, without departing from the spirit and scope thereof, can make various changes and modifications to adapt it to various usages and conditions.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described in the foregoing paragraphs. In addition, the materials, methods, and examples are illustrative only and not intended to be limiting. In case of conflict, the present specification, including definitions, will control.

All United States patents and published or unpublished United States patent applications cited herein are incorporated by reference. All published foreign patents and patent applications cited herein are hereby incorporated by reference. All published references, documents, manuscripts, scientific literature cited herein are hereby incorporated by reference. All identifier and accession numbers pertaining to scientific databases referenced herein (e.g., PUBMED, NCBI) are hereby incorporated by reference.

1. A computer readable medium comprising computer-executable instructions, which, when executed by a processor, cause the processor to carry out a method or a set of steps for diagnosing dementia in a subject, the method or steps comprising,
a) extracting, into a diagnostic model, a plurality of features comprising
1) structural features of a brain tissue of the subject or a region thereof;
2) genetic features from the subject's biological sample;
3) optionally actionable risk features; and
4) further optionally epidemiological features;
b) mathematically integrating the structural features and the genetic features in the diagnostic model to output a first integrated score;
c) optionally integrating actionable risk features in the diagnostic model to output a second integrated score and/or further integrating epidemiological features in the diagnostic model to output a third integrated score and outputting a risk score based on the first, second or third integrated scores; and
d) diagnosing dementia based on the risk score.

2. The computer readable medium of claim 1, wherein the processor to carries out a method or a set of steps for diagnosing dementia in a subject, the method or steps comprising,
a) extracting, into the diagnostic model, a plurality of features comprising the structural features, the genetic features and the actionable risk features;
b) mathematically integrating the structural features and the genetic features to output a first integrated score;
c) further integrating actionable risk features in the diagnostic model to output a second integrated score and outputting a risk score based on the second integrated score; and
d) diagnosing dementia based on the risk score.

3. The computer readable medium of claim 1, wherein the processor to carries out a method or a set of steps for diagnosing dementia in a subject, the method or steps comprising,
a) extracting, into the diagnostic model, a plurality of features comprising the structural features, the genetic features, the actionable risk features, and the epidemiological features;
b) mathematically integrating the structural features and the genetic features to output a first integrated score;
c) further integrating actionable risk features in the diagnostic model to output a second integrated score and integrating the epidemiological features in the diagnostic model to output a third integrated score and outputting a risk score based on the third integrated score; and
d) diagnosing dementia based on the risk score.

4. The computer readable medium of claim 1, wherein the genetic features comprises at least 1, 2, 3, 4, 5, 6, 7, 8, 9, 10, 11, 12, 13, 14, 15, 16, 17, 18, 19, 20, 21, or all of the genetic features of Table 1
TABLE 1
List of genetic features associated with dementia,
in decreasing order of relevance to the risk score

| Chromosome | Region_Start | Region_Stop |
|---|---|---|
| chr19 | 43908684 | 45908684 |
| chr2 | 25135287 | 27135287 |
| chr11 | 120564878 | 122564878 |
| chr2 | 126135234 | 128135234 |
| chr1 | 206518704 | 208518704 |
| chr8 | 26610169 | 28610169 |
| chr19 | 63444 | 2063444 |
| chr11 | 85156833 | 87156833 |
| chr14 | 51933911 | 53933911 |
| chr20 | 55443204 | 57443204 |
| chr11 | 59156035 | 61156035 |
| chr7 | 142413669 | 144413669 |
| chr6 | 31610753 | 33610753 |
| chr6 | 46520026 | 48520026 |
| chr8 | 26337604 | 28337604 |
| chr14 | 91460608 | 93460608 |
| chr7 | 99406823 | 101406823 |
| chr11 | 46536319 | 48536319 |
| chr2 | 232159830 | 234159830 |
| chr5 | 87927603 | 89927603 |
| chr7 | 36801932 | 38801932 |

5. The computer readable medium of claim 1, wherein the genetic features comprises at least 1, 2, 3, 4, 5, 6, 7, 8, 9, 10, 11, 12, 13, 14, 15, 16, 17, 18, 19, 20, or more SNPs or a locus related thereto.

6. The computer readable medium of claim 1, wherein the genetic features comprises at least 1, 2, 3, 4, 5, 6, 7, 8, 9, 10, 11, 12, 13, 14, 15, 16, 17, 18, 19, 20, or all of the SNPs having the Ref SNP ID Nos. rs429358; rs11218343; rs6733839; rs6656401; rs9331896; rs4147929; rs10792832; rs17125944; rs7274581; rs983392; rs11771145; rs9271192; rs10948363; rs28834970; rs10498633; rs1476679; rs10838725; rs35349669; rs190982; rs2718058 or a locus related thereto.

7. The computer readable medium of claim 1, wherein the genetic features comprises at least 1, 2, 3, 4, 5, 6, 7, 8, 9, 10, 11, 12, 13, 14, 15, 16, 17, 18, 19, 20, or all of the SNPs of Table 2 or a locus related thereto
TABLE 2
List of SNPs, ranked in decreasing order of effect size.

| rsID | Chromosome | Position |
|---|---|---|
| rs429358 | chr19 | 44908684 |
| rs11218343 | chr11 | 121564878 |
| rs6733839 | chr2 | 127135234 |
| rs6656401 | chr1 | 207518704 |
| rs9331896 | chr8 | 27610169 |
| rs4147929 | chr19 | 1063444 |
| rs10792832 | chr11 | 86156833 |
| rs17125944 | chr14 | 52933911 |
| rs7274581 | chr20 | 56443204 |
| rs983392 | chr11 | 60156035 |
| rs11771145 | chr7 | 143413669 |
| rs9271192 | chr6 | 32610753 |
| rs10948363 | chr6 | 47520026 |
| rs28834970 | chr8 | 27337604 |
| rs10498633 | chr14 | 92460608 |
| rs1476679 | chr7 | 100406823 |
| rs10838725 | chr11 | 47536319 |
| rs35349669 | chr2 | 233159830 |
| rs190982 | chr5 | 88927603 |
| rs2718058 | chr7 | 37801932 |

8. The computer readable medium of claim 1, wherein the genetic features comprises at least 1, 2, 3, 4, 5, 6, 7 or all of the rare SNPs having the Ref SNP ID Nos. rs202198008; rs538591288; rs148046938; rs113809142; rs201060968; rs775332895; and/or rs76763715 or a locus related thereto.

9. The computer readable medium of claim 1, wherein the genetic features comprises at least 1, 2, 3, 4, 5, 6, 7 or all of the rare SNPs are selected from the SNPs of Table 3 or a locus related thereto
TABLE 3
Rare genetic markers associated with dementia

| Chromosome | Position | dbSNP | Gene | ExAC AF |
|---|---|---|---|---|
| chr21 | 26021879 | rs202198008 | APP | 0.0006 |
| chr19 | 1055908 | rs538591288 | ABCA7 | 0.000882 |
| chr19 | 15186898 | rs148046938 | NOTCH3 | 0.000701 |
| chr19 | 1056245 | rs113809142 | ABCA7 | 0.000156 |
| chr19 | 1054256 | rs201060968 | ABCA7 | 0.000518 |
| chr22 | 23767396 | rs775332895 | CHCHD10 | 0.000287 |
| chr1 | 1.55E+08 | rs76763715 | GBA | 0.00221 |

10. The computer readable medium of claim 1, wherein the genetic features comprise genetic variations comprising SNPs and/or CNVs the method includes calculation of a polygenic risk score.

11. The computer readable medium of claim 1, wherein the polygenic risk score is calculated by summation of the number of risk alleles carried by an individual for each variant, weighted by the effect size (log₂(OR)) from a genome-wide association study.

12. The computer readable medium of claim 1, wherein the structural features of brain tissue comprises magnetic resonance imaging (MRI) data.

13. The computer readable medium of claim 1, wherein the structural features include volume, cortical thickness, and cortical surface area, which are extracted for regions known to have an effect size greater than 1.

14. The computer readable medium of claim 1, wherein the structural feature of brain tissue comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or all of the image features of Table 4
TABLE 4
List of image features

| SN | Image feature |
|---|---|
| 1 | Estimated Total IntraCranial Volume |
| 2 | Left hemisphere Hippocampus Volume |
| 3 | Right hemisphere Hippocampus Volume |
| 4 | Left hemisphere Lateral Ventricle Volume |
| 5 | Right hemisphere Lateral Ventricle Volume |
| 6 | Left hemisphere Inferior Lateral Ventricle Volume |
| 7 | Right hemisphere Inferior Lateral Ventricle Volume |
| 8 | Left hemisphere Amygdala Volume |
| 9 | Right hemisphere Amygdala Volume |
| 10 | Left hemisphere entorlinal Gray Volume |
| 11 | Left hemisphere entorlinal Surface Area |
| 12 | Left hemisphere entorlinal Thickness Average |
| 13 | Right hemisphere entorlinal Gray Volume |
| 14 | Right hemisphere entorlinal Surface Area |
| 15 | Right hemisphere entorlinal Thickness Average |
| 16 | Left hemisphere parahippocampal Gray Volume |
| 17 | Right hemisphere parahippocampal Gray Volume |
| 18 | Left hemisphere inferiorparietal Gray Volume |
| 19 | Left hemisphere inferiorparietal Surface Area |
| 20 | Left hemisphere inferiorparietal Thickness Average |
| 21 | Right hemisphere inferiorparietal Gray Volume |
| 22 | Right hemisphere inferiorparietal Surface Area |
| 23 | Right hemisphere inferiorparietal Thickness Average |
| 24 | Left hemisphere rostral middle frontal Gray Volume |
| 25 | Left hemisphere rostral middle frontal Surface Area |
| 26 | Left hemisphere rostral middle frontal Thickness Average |
| 27 | Right hemisphere rostral middle frontal Gray Volume |
| 28 | Right hemisphere rostral middle frontal Surface Area |
| 29 | Right hemisphere rostral middle frontal Thickness Average |
| 30 | Left hemisphere isthmuscingulate Gray Volume |
| 31 | Left hemisphere isthmuscingulate Surface Area |
| 32 | Left hemisphere isthmuscingulate Thickness Average |
| 33 | Right hemisphere isthmuscingulate Gray Volume |
| 34 | Right hemisphere isthmuscingulate Surface Area |
| 35 | Right hemisphere isthmuscingulate Thickness Average |
| 36 | Left hemisphere supramarginal Gray Volume |
| 37 | Left hemisphere supramarginal Surface Area |
| 38 | Left hemisphere supramarginal Thickness Average |
| 39 | Right hemisphere supramarginal Gray Volume |
| 40 | Right hemisphere supramarginal Surface Area |
| 41 | Right hemisphere supramarginal Thickness Average |
| 42 | Left hemisphere caudal middle frontal Gray Volume |
| 43 | Left hemisphere caudal middle frontal Surface Area |
| 44 | Left hemisphere caudal middle frontal Thickness Average |
| 45 | Right hemisphere caudal middle frontal Gray Volume |
| 46 | Right hemisphere caudal middle frontal Surface Area |
| 47 | Right hemisphere caudal middle frontal Thickness Average |
| 48 | Left hemisphere fusiform Gray Volume |
| 49 | Left hemisphere fusiform Surface Area |
| 50 | Left hemisphere fusiform Thickness Average |
| 51 | Right hemisphere fusiform Gray Volume |
| 52 | Right hemisphere fusiform Surface Area |
| 53 | Right hemisphere fusiform Thickness Average |
| 54 | Right hemisphere middle temporal Gray Volume |
| 55 | Left hemisphere middle temporal Gray Volume |
| 56 | Right hemisphere middle temporal Surface Area |
| 57 | Left hemisphere middle temporal Surface Area |
| 58 | Right hemisphere middle temporal Thickness Average |
| 59 | Left hemisphere middle temporal Thickness Average |
| 60 | Right hemisphere inferior temporal Gray Volume |
| 61 | Left hemisphere inferior temporal Gray Volume |
| 62 | Right hemisphere inferior temporal Surface Area |
| 63 | Left hemisphere inferior temporal Surface Area |
| 64 | Right hemisphere inferior temporal Thickness Average |
| 65 | Left hemisphere inferior temporal Thickness Average |
| 66 | Right hemisphere parahippocampal Surface Area |
| 67 | Left hemisphere parahippocampal Surface Area |
| 68 | Right hemisphere parahippocampal Thickness Average |
| 69 | Left hemisphere parahippocampal Thickness Average |
| 70 | Right hemisphere precuneus Gray Volume |
| 71 | Left hemisphere precuneus Gray Volume |
| 72 | Right hemisphere precuneus Surface Area |
| 73 | Left hemisphere precuneus Surface Area |
| 74 | Right hemisphere precuneus Thickness Average |
| 75 | Left hemisphere precuneus Thickness Average |
| 76 | Left hemisphere Hippocampal occupancy |
| 77 | Right hemisphere Hippocampal occupancy |
| 78 | White Matter hypointensities from T1W imaging |
| 79 | White Matter hyperintensities from FLAIR (volume, count, location) |

15. The computer readable medium of claim 1, wherein the structural features are integrated with genetic features using machine learning which comprises (1) a regularized linear model, (2) an ensemble model using boosted trees, or (3) a neural network (long short-term memory or LSTM).

16. The computer readable medium of claim 1, wherein the mathematical integration comprises concatenation of the structural features with the genetic features using long short-term memory neural network.

17. The computer readable medium of claim 1, wherein the actionable risk features comprise alcohol use, obesity, diabetes, high blood pressure, high cholesterol, vitamin B12, depression, head injuries, and lack of physical activity; preferably, high BMI, alcohol abuse, high cortisol, low vitamin B12, high medium-chain triglycerides (MCTs), elevated bilirubin, high triglyceride level, high serum uric acid, high diastolic blood pressure, and high systolic blood pressure.

18. The computer readable medium of claim 1, wherein the epidemiological risk features comprise age-specific and gender-specific population incidence rates of dementia.

19. A system for diagnosing dementia, comprising,
a) a receiver for receiving a plurality of features comprising
1) structural features of a brain tissue of the subject or a region thereof;
2) genetic features from the subject's biological sample;
3) optionally actionable risk features; and
4) further optionally epidemiological features;
b) a first integrator for integrating structural features and genetic features to output a first integrated score;
c) an optional second integrator for integrating actionable risk features in the diagnostic model to output a second integrated score and a further optional third integrator for integrating the epidemiological features in the diagnostic model to output a third integrated score; and
d) a scorer for determining a risk of dementia based on the first, second or third integrated score, wherein the risk score is used to diagnose dementia.

20. The system of claim 19, which comprises the second integrator.

21. The system of claim 19, which comprises the second integrator and the third integrator.

22. The system of claim 19, which further comprises (e) a reporter which generates a summary report of the subject's overall risk for developing dementia in the subject's lifetime and lists all the contributing factors to the risk.

23. A method for diagnosing dementia in a subject, comprising,
a) extracting, into a diagnostic model, a plurality of features comprising
1) structural features of a brain tissue of the subject or a region thereof;
2) genetic features from the subject's biological sample;
3) optionally actionable risk features; and
4) further optionally epidemiological features;
b) mathematically integrating the structural features and the genetic features in the diagnostic model to output a first integrated score;
c) optionally integrating actionable risk features in the diagnostic model to output a second integrated score and/or further integrating epidemiological features in the diagnostic model to output a third integrated score and outputting a risk score based on the first, second or third integrated scores; and
d) diagnosing dementia based on the risk score.

24. The method of claim 23, comprising,
a) extracting, into the diagnostic model, a plurality of features comprising the structural features, the genetic features and the actionable risk features;
b) mathematically integrating the structural features and the genetic features to output a first integrated score;
c) further integrating actionable risk features in the diagnostic model to output a second integrated score and outputting a risk score based on the second integrated score; and
d) diagnosing dementia based on the risk score.

25. The method of claim 23, comprising,
a) extracting, into the diagnostic model, a plurality of features comprising the structural features, the genetic features, the actionable risk features, and the epidemiological features;
b) mathematically integrating the structural features and the genetic features to output a first integrated score;
c) further integrating actionable risk features in the diagnostic model to output a second integrated score and integrating the epidemiological features in the diagnostic model to output a third integrated score and outputting a risk score based on the third integrated score; and
d) diagnosing dementia based on the risk score.

26. The method of claim 23, wherein the genetic features comprises at least 1, 2, 3, 4, 5, 6, 7, 8, 9, 10, 11, 12, 13, 14, 15, 16, 17, 18, 19, 20, 21, or all of the genetic features of Table 1
TABLE 1
List of genetic features associated with dementia, with
decreasing order of relevance to the risk score

| Chromosome | Region_Start | Region_Stop |
|---|---|---|
| chr19 | 43908684 | 45908684 |
| chr2 | 25135287 | 27135287 |
| chr11 | 120564878 | 122564878 |
| chr2 | 126135234 | 128135234 |
| chr1 | 206518704 | 208518704 |
| chr8 | 26610169 | 28610169 |
| chr19 | 63444 | 2063444 |
| chr11 | 85156833 | 87156833 |
| chr14 | 51933911 | 53933911 |
| chr20 | 55443204 | 57443204 |
| chr11 | 59156035 | 61156035 |
| chr7 | 142413669 | 144413669 |
| chr6 | 31610753 | 33610753 |
| chr6 | 46520026 | 48520026 |
| chr8 | 26337604 | 28337604 |
| chr14 | 91460608 | 93460608 |
| chr7 | 99406823 | 101406823 |
| chr11 | 46536319 | 48536319 |
| chr2 | 232159830 | 234159830 |
| chr5 | 87927603 | 89927603 |
| chr7 | 36801932 | 38801932 |

27. The method of claim 23, wherein the genetic features comprises at least 1, 2, 3, 4, 5, 6, 7, 8, 9, 10, 11, 12, 13, 14, 15, 16, 17, 18, 19, 20, or more SNPs or a locus related thereto.

28. The method of claim 23, wherein the genetic features comprises at least 1, 2, 3, 4, 5, 6, 7, 8, 9, 10, 11, 12, 13, 14, 15, 16, 17, 18, 19, 20, or all of the SNPs having the Ref SNP ID Nos. rs429358; rs11218343; rs6733839; rs6656401; rs9331896; rs4147929; rs10792832; rs17125944; rs7274581; rs983392; rs11771145; rs9271192; rs10948363; rs28834970; rs10498633; rs1476679; rs10838725; rs35349669; rs190982; rs2718058 or a locus related thereto.

29. The method of claim 23, wherein the genetic features comprises at least 1, 2, 3, 4, 5, 6, 7, 8, 9, 10, 11, 12, 13, 14, 15, 16, 17, 18, 19, 20, or all of the SNPs of Table 2 or a locus related thereto
TABLE 2
List of SNPs, ranked in decreasing order of effect size.

| rsID | Chromosome | Position |
|---|---|---|
| rs429358 | chr19 | 44908684 |
| rs11218343 | chr11 | 121564878 |
| rs6733839 | chr2 | 127135234 |
| rs6656401 | chr1 | 207518704 |
| rs9331896 | chr8 | 27610169 |
| rs4147929 | chr19 | 1063444 |
| rs10792832 | chr11 | 86156833 |
| rs17125944 | chr14 | 52933911 |
| rs7274581 | chr20 | 56443204 |
| rs983392 | chr11 | 60156035 |
| rs11771145 | chr7 | 143413669 |
| rs9271192 | chr6 | 32610753 |
| rs10948363 | chr6 | 47520026 |
| rs28834970 | chr8 | 27337604 |
| rs10498633 | chr14 | 92460608 |
| rs1476679 | chr7 | 100406823 |
| rs10838725 | chr11 | 47536319 |
| rs35349669 | chr2 | 233159830 |
| rs190982 | chr5 | 88927603 |
| rs2718058 | chr7 | 37801932 |

30. The method of claim 23, wherein the genetic features comprises at least 1, 2, 3, 4, 5, 6, 7 or all of the rare SNPs having the Ref SNP ID Nos. rs202198008; rs538591288; rs148046938; rs113809142; rs201060968; rs775332895; and/or rs76763715 or a locus related thereto.

31. The method of claim 23, wherein the genetic features comprises at least 1, 2, 3, 4, 5, 6, 7 or all of the rare SNPs are selected from the SNPs of Table 3 or a locus related thereto
TABLE 3
Rare genetic markers associated with dementia

| Chromosome | Position | dbSNP | Gene | ExAC AF |
|---|---|---|---|---|
| chr21 | 26021879 | rs202198008 | APP | 0.0006 |
| chr19 | 1055908 | rs538591288 | ABCA7 | 0.000882 |
| chr19 | 15186898 | rs148046938 | NOTCH3 | 0.000701 |
| chr19 | 1056245 | rs113809142 | ABCA7 | 0.000156 |
| chr19 | 1054256 | rs201060968 | ABCA7 | 0.000518 |
| chr22 | 23767396 | rs775332895 | CHCHD10 | 0.000287 |
| chr1 | 1.55E+08 | rs76763715 | GBA | 0.00221 |

32. The method of claim 23, wherein the genetic features comprise genetic variations comprising SNPs and/or CNVs the method includes calculation of a polygenic risk score.

33. The method of claim 23, wherein the polygenic risk score is calculated by summation of the number of risk alleles carried by an individual for each variant, weighted by the effect size (log₂(OR)) from a genome-wide association study.

34. The method of claim 23, wherein the structural features of brain tissue comprises magnetic resonance imaging (MRI) data.

35. The method of claim 23, wherein the structural features include volume, cortical thickness, and cortical surface area, which are extracted for regions known to have an effect size greater than 1.

36. The method of claim 23, wherein the structural feature of brain tissue comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or all of the image features of Table 4
TABLE 4
List of image features

| SN | Image feature |
|---|---|
| 1 | Estimated Total IntraCranial Volume |
| 2 | Left hemisphere Hippocampus Volume |
| 3 | Right hemisphere Hippocampus Volume |
| 4 | Left hemisphere Lateral Ventricle Volume |
| 5 | Right hemisphere Lateral Ventricle Volume |
| 6 | Left hemisphere Inferior Lateral Ventricle Volume |
| 7 | Right hemisphere Inferior Lateral Ventricle Volume |
| 8 | Left hemisphere Amygdala Volume |
| 9 | Right hemisphere Amygdala Volume |
| 10 | Left hemisphere entorlinal Gray Volume |
| 11 | Left hemisphere entorlinal Surface Area |
| 12 | Left hemisphere entorlinal Thickness Average |
| 13 | Right hemisphere entorlinal Gray Volume |
| 14 | Right hemisphere entorlinal Surface Area |
| 15 | Right hemisphere entorlinal Thickness Average |
| 16 | Left hemisphere parahippocampal Gray Volume |
| 17 | Right hemisphere parahippocampal Gray Volume |
| 18 | Left hemisphere inferiorparietal Gray Volume |
| 19 | Left hemisphere inferiorparietal Surface Area |
| 20 | Left hemisphere inferiorparietal Thickness Average |
| 21 | Right hemisphere inferiorparietal Gray Volume |
| 22 | Right hemisphere inferiorparietal Surface Area |
| 23 | Right hemisphere inferiorparietal Thickness Average |
| 24 | Left hemisphere rostral middle frontal Gray Volume |
| 25 | Left hemisphere rostral middle frontal Surface Area |
| 26 | Left hemisphere rostral middle frontal Thickness Average |
| 27 | Right hemisphere rostral middle frontal Gray Volume |
| 28 | Right hemisphere rostral middle frontal Surface Area |
| 29 | Right hemisphere rostral middle frontal Thickness Average |
| 30 | Left hemisphere isthmuscingulate Gray Volume |
| 31 | Left hemisphere isthmuscingulate Surface Area |
| 32 | Left hemisphere isthmuscingulate Thickness Average |
| 33 | Right hemisphere isthmuscingulate Gray Volume |
| 34 | Right hemisphere isthmuscingulate Surface Area |
| 35 | Right hemisphere isthmuscingulate Thickness Average |
| 36 | Left hemisphere supramarginal Gray Volume |
| 37 | Left hemisphere supramarginal Surface Area |
| 38 | Left hemisphere supramarginal Thickness Average |
| 39 | Right hemisphere supramarginal Gray Volume |
| 40 | Right hemisphere supramarginal Surface Area |
| 41 | Right hemisphere supramarginal Thickness Average |
| 42 | Left hemisphere caudal middle frontal Gray Volume |
| 43 | Left hemisphere caudal middle frontal Surface Area |
| 44 | Left hemisphere caudal middle frontal Thickness Average |
| 45 | Right hemisphere caudal middle frontal Gray Volume |
| 46 | Right hemisphere caudal middle frontal Surface Area |
| 47 | Right hemisphere caudal middle frontal Thickness Average |
| 48 | Left hemisphere fusiform Gray Volume |
| 49 | Left hemisphere fusiform Surface Area |
| 50 | Left hemisphere fusiform Thickness Average |
| 51 | Right hemisphere fusiform Gray Volume |
| 52 | Right hemisphere fusiform Surface Area |
| 53 | Right hemisphere fusiform Thickness Average |
| 54 | Right hemisphere middle temporal Gray Volume |
| 55 | Left hemisphere middle temporal Gray Volume |
| 56 | Right hemisphere middle temporal Surface Area |
| 57 | Left hemisphere middle temporal Surface Area |
| 58 | Right hemisphere middle temporal Thickness Average |
| 59 | Left hemisphere middle temporal Thickness Average |
| 60 | Right hemisphere inferior temporal Gray Volume |
| 61 | Left hemisphere inferior temporal Gray Volume |
| 62 | Right hemisphere inferior temporal Surface Area |
| 63 | Left hemisphere inferior temporal Surface Area |
| 64 | Right hemisphere inferior temporal Thickness Average |
| 65 | Left hemisphere inferior temporal Thickness Average |
| 66 | Right hemisphere parahippocampal Surface Area |
| 67 | Left hemisphere parahippocampal Surface Area |
| 68 | Right hemisphere parahippocampal Thickness Average |
| 69 | Left hemisphere parahippocampal Thickness Average |
| 70 | Right hemisphere precuneus Gray Volume |
| 71 | Left hemisphere precuneus Gray Volume |
| 72 | Right hemisphere precuneus Surface Area |
| 73 | Left hemisphere precuneus Surface Area |
| 74 | Right hemisphere precuneus Thickness Average |
| 75 | Left hemisphere precuneus Thickness Average |
| 76 | Left hemisphere Hippocampal occupancy |
| 77 | Right hemisphere Hippocampal occupancy |
| 78 | White Matter hypointensities from T1W imaging |
| 79 | White Matter hyperintensities from FLAIR (volume, count, location) |

37. The method of claim 23, wherein the structural features are integrated with genetic features using machine learning which comprises (1) a regularized linear model, (2) an ensemble model using boosted trees, or (3) a neural network (long short-term memory or LSTM).

38. The method of claim 23, wherein the mathematical integration comprises concatenation of the structural features with the genetic features using long short-term memory neural network.

39. The method of claim 23, wherein the actionable risk features comprise alcohol use, obesity, diabetes, high blood pressure, high cholesterol, vitamin B12, depression, head injuries, and lack of physical activity; preferably, high BMI, alcohol abuse, high cortisol, low vitamin B12, high medium-chain triglycerides (MCTs), elevated bilirubin, high triglyceride level, high serum uric acid, high diastolic blood pressure, and high systolic blood pressure.

40. The method of claim 23, wherein the epidemiological risk features comprise age-specific and gender-specific population incidence rates of dementia.

41. The method of claim 1, further comprising determining short-term or long-term risk; personalizing risk using annualized incidence rates; determining disease trajectory; identifying short-term risk of memory decline; and recommending an action with a recommender.
